(19) United States
(12) Patent Application Publication
(10) Pub. No.: US 2024/0082399 A1
(43) Pub. Date: Mar. 14, 2024

(54) RECOMBINANT POLYPEPTIDES FOR REGULATABLE CELLULAR LOCALIZATION

(71) Applicant: The Board of Trustees of the Leland Stanford Junior University, Stanford, CA (US)

(72) Inventors: Crystal Mackall, Stanford, CA (US); Robbie Majzner, Palo Alto, CA (US); Louai Labanieh, Palo Alto, CA (US); Michael Lin, Stanford, CA (US)

(21) Appl. No.: 17/767,818

(22) PCT Filed: Oct. 9, 2020
(86) PCT No.: PCT/US2020/055057

§ 371 Date: Apr. 8, 2022

Related U.S. Application Data
(60) Provisional application No. 62/914,310, filed on Oct. 11, 2019.

(51) Int. Cl. A61K 39/00; C07K 14/705; C07K 14/725; C07K 16/28; C07K 16/30; C07K 16/32; C12N 5/0783

(52) U.S. Cl. CPC A61K 39/4631; A61K 39/4611; A61K 39/4632; C07K 14/7051; C07K 14/70521; C07K 16/2803; C07K 16/2827; C07K 16/3084; C07K 16/32; C12N 5/0636; A61K 2239/23; C07K 2317/622; C07K 2319/03; C07K 2319/04; C07K 2319/05; C07K 2319/06; C07K 2319/50; C07K 2319/60; C12N 2510/00

(57) ABSTRACT

Provided are recombinant polypeptides that comprise a protein of interest, a protein localization tag, and a protease cleavage site disposed between the protein of interest and the protein localization tag. In certain embodiments, the recombinant polypeptides further comprise a protease, where the protease cleavage site is a cleavage site for the protease. Also provided are nucleic acids that encode the recombinant polypeptides, cells that comprise such nucleic acids, and compositions (e.g., pharmaceutical compositions) that comprise such cells. Methods of regulating cellular localization of a protein of interest, and methods of administering a regulatable cell-based therapy to an individual in need thereof, are also provided.

### CROSS-REFERENCE TO RELATED APPLICATIONS

This application claims the benefit of U.S. Provisional Patent Application No. 62/914,310, filed Oct. 11, 2019, which application is incorporated herein by reference in its entirety.

### SUMMARY

Provided are recombinant polypeptides that comprise a protein of interest, a protein localization tag, and a protease cleavage site disposed between the protein of interest and the protein localization tag. In certain embodiments, the recombinant polypeptides further comprise a protease, where the protease cleavage site is a cleavage site for the protease.

Also provided are nucleic acids that encode the recombinant polypeptides, cells that comprise such nucleic acids, and compositions (e.g., pharmaceutical compositions) that comprise such cells. Methods of regulating cellular localization of a protein of interest and methods of administering a regulatable cell-based therapy to an individual in need thereof are also provided.

### BRIEF DESCRIPTION OF THE FIGURES

FIG. 1: Panel A: A schematic illustration of a recombinant polypeptide according to an embodiment of the present disclosure. The recombinant polypeptide comprises a protein of interest, a protease cleavage site (“cleavage site”), and a protein localization tag (referred to in FIG. 1 as a “retention signal”). In this example, the protein of interest is a chimeric antigen receptor (CAR). Also in this example, the recombinant polypeptide further comprises a protease, where the protease cleavage site is a cleavage site for the protease incorporated in the recombinant polypeptide. The combination of the protease cleavage site and protein localization tag (and if present, a protease that cleaves the protease cleavage site) are sometimes referred to herein as a “STASH tag”, because the recombinant construct is designed for intracellular storage by targeted shuttling. Panel B: Schematic illustration of regulatable cellular localization of the CAR of the recombinant polypeptide shown in panel A. In this example, the protein localization tag is an endoplasmic reticulum (ER) localization tag that directs localization of the recombinant polypeptide to the ER. In the absence of an inhibitor of the protease (“−drug”, left), the protease cleaves the protease cleavage site, thereby removing the ER localization tag from the CAR, and enabling expression (and activity) of the CAR on the surface of the cell. In the presence of an inhibitor of the protease (“+drug”, right), the protease does not cleave the protease cleavage site, so the ER localization tag remains attached to the CAR and the CAR is retained at the ER.

FIG. 2: Cell surface expression of a CAR comprising different protein localization tags targeting the endoplasmic reticulum (ER variant 1, ER variant 2), the Golgi apparatus (Golgi), or the lysosome (lysosome) in the presence and absence of an inhibitor of the protease, as determined by flow cytometry.

FIG. 3: Panel A: A graph showing the quantification of CAR molecules on the cell surface by flow cytometry tested in the presence (+) or absence (−) of an inhibitor of the protease (“drug”). Panel B: A graph showing the reduction of CAR surface expression over time after incubation with an inhibitor of the protease.

FIG. 4: Panel A: A schematic illustration of recombinant polypeptide according to an embodiment of the present disclosure. The recombinant polypeptide comprises a CAR as the protein of interest, and an ER localization tag (“ER tag”) as the protein localization tag. Panel B: Schematic illustration of a cell labeled with fluorescently tagged proteins that localize to various cellular compartments.

FIG. 5: Fluorescence microscopy images of human 293T cells (derived from the HEK 293 cell line) transduced with the recombinant polypeptide shown in FIG. 4 (comprising an ER localization tag) and incubated in the absence (−drug) or presence (+drug) of an inhibitor of the protease.

FIG. 6: Panel A: A schematic illustration of CAR- and GFP-containing recombinant polypeptides localized at the cell surface or localized intracellularly and their corresponding staining, where “+” represents positive staining and “−” represents negative staining. Panel B: A plot of the quantification of flow cytometry data for cells expressing the recombinant polypeptides that have been incubated with an inhibitor of the protease for various amounts of time.

FIG. 7: Panel A: A graph showing the GFP fluorescence of GFP-expressing D425 medulloblastoma cells which also express the B7H3 antigen. The tumor cells were co-cultured with B7H3-CAR-STASH T cells in the presence (+drug) or absence (−drug) of an inhibitor of the protease. Panel B: A graph showing quantification of interferon gamma (IFNγ) levels in co-culture supernatant taken from co-cultures described in FIG. 7, panel A.

FIG. 8: Schematic illustrations of recombinant polypeptides of the present disclosure comprising a membrane protein (in this example, a CAR) and an ER localization tag. Shown on the left is a configuration in which the recombinant polypeptide further comprises the protease—referred to herein as a “cis” configuration. Shown on the right is a configuration in which the recombinant polypeptide does not comprise the protease, and where the protease is provided tethered to the membrane in sufficient proximity to the cleavage site of the recombinant polypeptide such that in the absence of an inhibitor of the protease, the protease cleaves the protease cleavage site—referred to herein as a “trans” configuration. In both configurations, cleavage of the protease cleavage site results in the CAR being expressed on the surface of the cell and no longer retained at the ER.

FIG. 9: Schematic illustrations of recombinant polypeptides of the present disclosure comprising a secreted effector protein (e.g., a cytokine, chemokine, growth factor, or the like) and an ER localization tag. In this example, prior to protease cleavage (“+drug”), the secreted effector protein, protease cleavage site and protease are located in the lumen of the ER. Shown on the left is the cis configuration in which the recombinant polypeptide further comprises the protease. Shown on the right is the trans configuration in which the recombinant polypeptide does not comprise the protease, and where the protease is provided tethered to the membrane on the ER lumen side in sufficient proximity to the cleavage site of the recombinant polypeptide such that in the absence of an inhibitor of the protease, the protease cleaves the protease cleavage site. In both configurations, cleavage of the protease cleavage site results in the effector molecule becoming soluble in the ER lumen and then secreted into the extracellular space.

### DETAILED DESCRIPTION

Before the recombinant polypeptides, methods and other aspects of the present disclosure are described in greater detail, it is to be understood that the recombinant polypeptides, methods and other aspects of the present disclosure are not limited to particular exemplary embodiments described herein, as such may, of course, vary. It is also to be understood that the terminology used herein is for the purpose of describing particular exemplary embodiments only, and is not intended to be limiting, since the scope of the recombinant polypeptides, methods and other aspects will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the recombinant polypeptides, methods and other aspects. The upper and lower limits of these smaller ranges may independently be included in the smaller ranges and are also encompassed within the recombinant polypeptides, methods and other aspects, subject to any specifically excluded limit in the stated range. Where the stated range includes one or both of the limits, ranges excluding either or both of those included limits are also included in the recombinant polypeptides, methods and other aspects.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the recombinant polypeptides, methods and other aspects belong. Although any recombinant polypeptides, methods and other aspects similar or equivalent to those described herein can also be used in the practice or testing of the recombinant polypeptides, methods and other aspects, representative illustrative recombinant polypeptides, methods and other aspects are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present recombinant polypeptides, methods and other aspects are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the recombinant polypeptides, methods and other aspects, which are, for clarity, described in the context of separate embodiments, may also be provided in combination in a single embodiment. Conversely, various features of the recombinant polypeptides, methods and other aspects, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present recombinant polypeptides, methods and other aspects and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Recombinant Polypeptides

Aspects of the present disclosure include recombinant polypeptides. The recombinant polypeptides comprise a protein of interest, a protein localization tag, and a protease cleavage site disposed between the protein of interest and the protein localization tag. According to some embodiments, the recombinant polypeptides comprise from N-terminus to C-terminus: the protein of interest, the protease cleavage site, and the protein localization tag. In certain embodiments, the recombinant polypeptides comprise from N-terminus to C-terminus: the protein localization tag; the protease cleavage site; and the protein of interest.

As surprisingly demonstrated herein, the recombinant polypeptides enable regulatable cellular localization of the protein of interest depending upon the presence or absence of an inhibitor of the protease. That is, in the presence of an inhibitor of the protease, the protease does not cleave the protease cleavage site, such that the protein localization tag is not removed from the protein of interest and the protein of interest is retained at the cellular compartment determined by the protein localization tag. In the absence of an inhibitor of the protease, the protease cleaves the protease cleavage site, thereby removing the protein localization tag from the protein of interest, and enabling expression (and activity) of the protein of interest at its normal destination in the absence of the protein localization tag.

The recombinant polypeptides find use in a variety research and clinical applications. By way of example, in the context of adoptive cell therapy (ACT), the recombinant polypeptides enable the conditional localization of a receptor on the cell surface (e.g., a chimeric antigen receptor (CAR), an engineered T cell receptor (TCR), or the like) enabling the regulation of activity of the cells expressing the receptors, thereby providing an improved approach for the prevention or delay of the onset of cell exhaustion resulting from activity of the receptor on the cell surface (e.g., T cell exhaustion resulting from CAR activity), turning off the activity of the cells in the event of adverse side effects (e.g., cytokine release syndrome resulting from unrestricted antigen-driven proliferation of the cells), and/or the like.

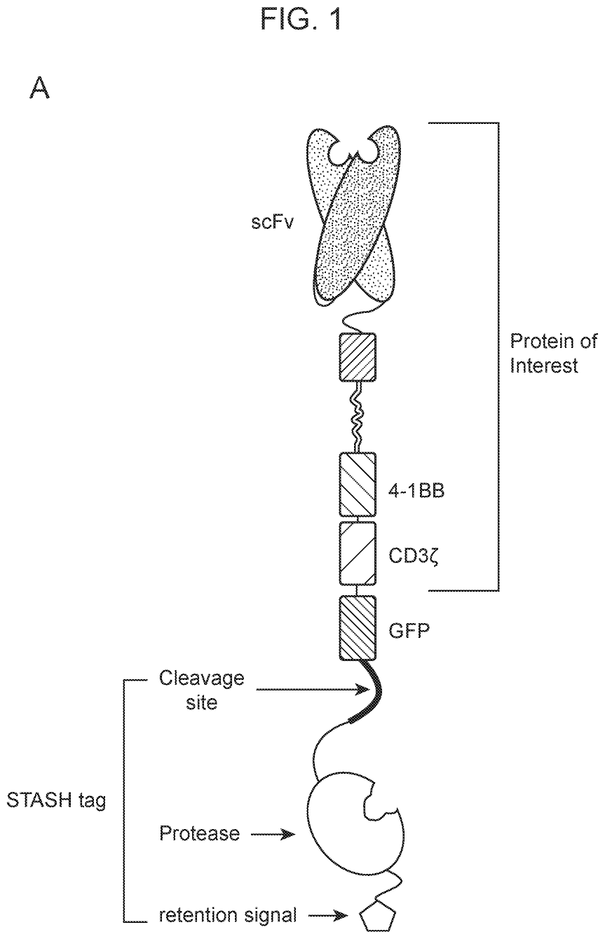

One example of such regulatable cellular localization is schematically illustrated in FIG. 1, panel B. In this example, the recombinant polypeptide comprises a chimeric antigen receptor (CAR) as the protein of interest and an endoplasmic reticulum (ER) localization tag (FIG. 1, panel A). As shown on the left of FIG. 1, panel B, in the absence of an inhibitor of the protease, the CAR is initially localized to the ER, and upon cleavage of the cleavage site by the protease, the CAR is no longer retained at the ER but rather is expressed on the surface of the cell (thereby becoming functional). As shown on the right of FIG. 1, panel B, in the presence of an inhibitor of the protease, the CAR remains associated with the ER localization tag and is therefore retained at the ER.

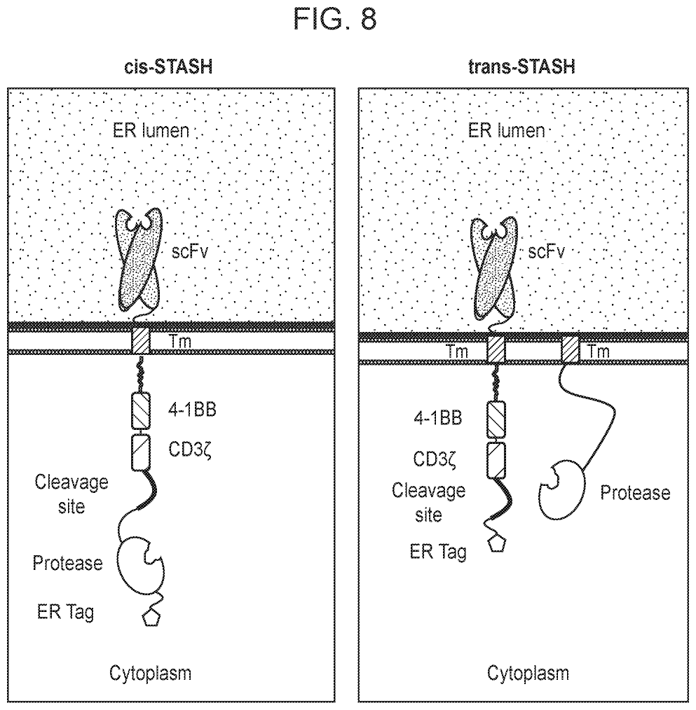

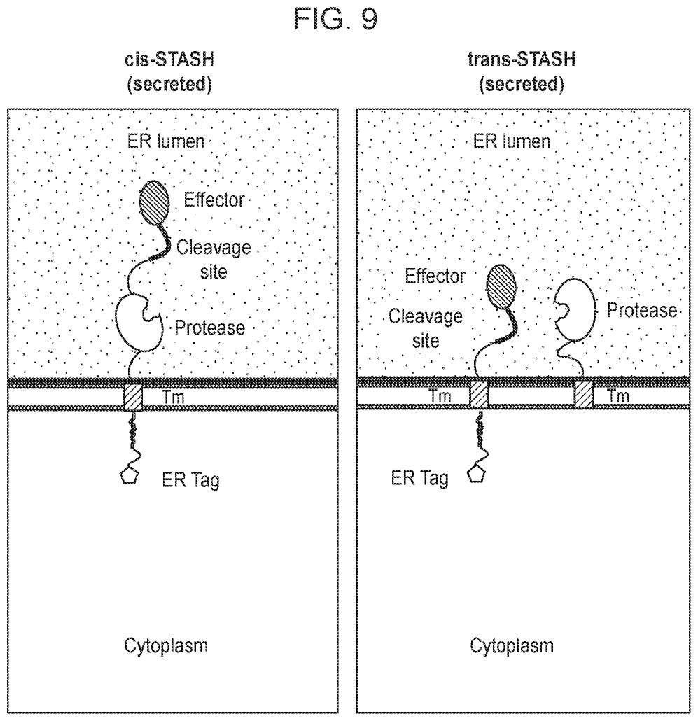

In certain embodiments, the polypeptides further comprise a protease (referred to herein as a “cis” configuration), where the protease cleavage site is a cleavage site for that same protease. According to some embodiments, the polypeptides do not comprise a protease that cleaves the protease cleavage site, but such a protease capable of cleaving the protease cleavage site is provided as a separate molecule—referred to herein as a “trans” configuration. Non-limiting examples of cis and trans configurations are schematically illustrated in FIGS. 8 and 9. Examples of proteases and protease cleavage sites that may be employed are described in more detail below.

A polypeptide of the present disclosure is recombinant. The term “recombinant” refers to polypeptides comprising two or more domains heterologous to each other—that is, two or more domains that are not found in a single polypeptide in nature.

The recombinant polypeptides may include domains in addition to the protein of interest, the protein localization tag, the protease cleavage site, and if in the cis configuration, the protease. For example, the recombinant polypeptides may include a spacer domain between one or more of the protein localization tag, the protease cleavage site, and if in the cis configuration, the protease. In some embodiments, the spacer domain includes a linker, a reporter domain, or a combination thereof. Non-limiting examples of reporter domains include fluorescent proteins (e.g., green fluorescent protein) and bioluminescent proteins. In certain embodiments, the recombinant polypeptide includes a bioluminescent protein (a protein that is bioluminescent on its own or catalyzes the production of bioluminescence), and the bioluminescent protein is a luciferase, e.g., a nanoluciferase. According to some embodiments, when the recombinant polypeptide includes a reporter domain, the reporter domain is disposed between the protein of interest and the protease cleavage site.

A recombinant polypeptide of the present disclosure may include one or more domains useful for purification (e.g., a purification tag, such as a FLAG tag, HIS tag, and/or the like), a domain useful for detecting/imaging the protein of interest (e.g., a luciferase or other proteinaceous element that enables detection/imaging (e.g., in vivo imaging) of the protein of interest directly or indirectly), and/or the like. The various domains of the subject polypeptides are operably linked to one another, meaning that such domains are linked to one another and retain their respective functions.

Protein Localization Tags

As used herein, the term “protein localization tag” refers to an amino acid sequence that directs the cellular localization of the recombinant polypeptide (and in turn, the protein of interest) to a particular cellular compartment. In certain embodiments, the protein localization tag is selected from an endoplasmic reticulum (ER) localization tag, a Golgi apparatus (Golgi) localization tag, a lysosome localization tag, a plasma membrane localization tag, a mitochondria localization tag, a peroxisome localization tag, a cytosolic localization tag, and a nuclear localization tag.

The recombinant polypeptide may include any suitable protein localization tag for directing localization of the recombinant polypeptide to the desired cellular compartment. Suitable protein localization tags are known. In certain embodiments, a recombinant polypeptide of the present disclosure includes a protein localization tag in LocSigDB (a database of protein localization signals/tags available at genome.unmc.edu/LocSigDB/and described in Negi et al. (2015) Database, Volume 2015:1-7); DBSubLoc (a database of protein subcellular localization—available at bioinfo.tsinghua.edu.cn/dbsubloc.html); LOCATE (a mammalian protein subcellular localization database available at locate.imb.uq.edu.au); LocDB (a protein localization database available at rostlab.org/services/locDB); eSLDB (a eukaryotic subcellular localization database available at gper.biocomp.unibo.it/esldb); and/or any other convenient database of protein localization tags. According to some embodiments, the protein localization tag is located at the N-terminus of the recombinant polypeptide. For example, there are naturally-occurring N-terminal protein localization tags for type II membrane proteins (see, e.g., Schutz et al. (1994) EMBO J. 13(7):1696-1705) and other proteins.

According to some embodiments, the protein localization tag is an ER localization tag. A non-limiting example of an ER localization tag that may be included in a recombinant polypeptide of the present disclosure is an ER localization tag comprising 85% or greater, 90% or greater, or 100% amino acid sequence identity to the amino acid sequence LYKYKSRRSFIDEKKMP (SEQ ID NO:1). Another example of an ER localization tag that may be included in a recombinant polypeptide of the present disclosure is an ER localization tag comprising the amino acid sequence KKMP (SEQ ID NO:2). Additional examples of ER localization tags that may be included in a recombinant polypeptide of the present disclosure include ER localization tags comprising 85% or greater, 90% or greater, or 100% amino acid sequence identity to one of the following ER localization tags: AEKDEL (SEQ ID NO:3); EQKLISEEDLKDEL (SEQ ID NO:4); GGGGSGGGGSKDEL (SEQ ID NO:5); GGGGSGGGGSGGGGSGGGGSKDEL (SEQ ID NO:6); GGGGSGGGGSGGGGSGGGGSAEKDEL (SEQ ID NO:7); KYKSRRSFIEEKKMP (SEQ ID NO:8); LKYKSRRSFIEEKKMP (SEQ ID NO:9); LYKYKSRRSFIEEKKMP (SEQ ID NO:10); LYCKYKSRRSFIEEKKMP (SEQ ID NO:11); LYCNKYKSRRSFIEEKKMP (SEQ ID NO:12); LYKYKSRRSFIDEKKMP (SEQ ID NO:13); LYCNKYKSRRSFIDEKKMP (SEQ ID NO:14); LYEQKLISEEDLKYKSRRSFIEEKKMP (SEQ ID NO:15); LYCYPYDVPDYAKYKSRRSFIEEKKMP (SEQ ID NO:16); LYKKLETFKKTN (SEQ ID NO:17); LYEQKLISEEDLKKLETFKKTN (SEQ ID NO:18); LYYQRL (SEQ ID NO:19); LYEQKLISEEDLYQRL (SEQ ID NO:20); LYKRKIIAFALEGKRSKVTRRPKASDYQRL (SEQ ID NO:21); LYRNIKCD (SEQ ID NO:22); and LYEQKLISEEDLRNIKCD (SEQ ID NO:23)

In certain embodiments, the protein localization tag is a Golgi localization tag. A non-limiting example of a Golgi localization tag that may be included in a recombinant polypeptide of the present disclosure is a Golgi localization tag comprising the amino acid sequence YQRL (SEQ ID NO:24).

According to some embodiments, the protein localization tag is a lysosome localization tag. A non-limiting example of a lysosome localization tag that may be included in a recombinant polypeptide of the present disclosure is a lysosome localization tag comprising the amino acid sequence KFERQ (SEQ ID NO:25).

Proteins of Interest

The recombinant polypeptide may include any of a variety of proteins of interest. In certain embodiments, the recombinant polypeptide comprises a protein of interest that is engineered. By “engineered” is meant the protein of interest does not have a native/wild-type counterpart, e.g., by virtue of the protein of interest including one or more heterologous domains, an engineered or synthetic domain (e.g., an engineered extracellular binding domain in the case of cell surface molecule (e.g., a cell surface receptor), etc.), and/or the like. In certain embodiments, the protein of interest is an engineered cell surface receptor. Non-limiting examples of engineered cell surface receptors include chimeric receptors (e.g., chimeric antigen receptors (CARs)), engineered T cell receptors (TCRs) (e.g., having altered (or “engineered”) specificity and/or affinity for an antigen as compared to a counterpart wild-type TCR, having one or more polypeptides covalently or non-covalently bound (e.g., fused) to one another, and/or the like), chimeric cytokine receptors (CCRs), chimeric chemokine receptors, synthetic notch receptors (synNotch), and the like.

In certain embodiments, the protein of interest is not engineered—that is, the protein of interest has a native/wild-type counterpart. According to some embodiments, a non-engineered protein of interest is a non-engineered cell surface receptor. Non-limiting examples of cell surface receptors having native/wild-type counterparts include stem cell receptors, immune cell receptors (e.g., T cell receptors, B cell receptors, and the like), growth factor receptors, cytokine receptors, hormone receptors, receptor tyrosine kinases, immune receptors such as CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H3, a type I cytokine receptor such as Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-9 receptor, Interleukin-11 receptor, Interleukin-12 receptor, Interleukin-13 receptor, Interleukin-15 receptor, Interleukin-18 receptor, Interleukin-21 receptor, Interleukin-23 receptor, Interleukin-27 receptor, Erythropoietin receptor, GM-CSF receptor, G-CSF receptor, Growth hormone receptor, Prolactin receptor, Leptin receptor, Oncostatin M receptor, Leukemia inhibitory factor, a type II cytokine receptor such as interferon-alpha/beta receptor, interferon-gamma receptor, Interferon type III receptor, Interleukin-10 receptor, Interleukin-20 receptor, Interleukin-22 receptor, Interleukin-28 receptor, a receptor in the tumor necrosis factor receptor superfamily such as Tumor necrosis factor receptor 2 (16), Tumor necrosis factor receptor 1, Lymphotoxin beta receptor, OX40, CD40, Fas receptor, Decoy receptor 3, CD27, CD30, 4-11313, Decoy receptor 2, Decoy receptor 1, Death receptor 5, Death receptor 4, RANK, Osteoprotegerin, TWEAK receptor, TACT, BAFF receptor, Herpesvirus entry mediator, Nerve growth factor receptor, B-cell maturation antigen, Glucocorticoid-induced TNFR-related, TROY, Death receptor 6, Death receptor 3, Ectodysplasin A2 receptor, a chemokine receptor such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, ACKR1, ACKR2, ACKR3, ACKR4, CCRL2, a receptor in the epidermal growth factor receptor (EGFR) family, a receptor in the fibroblast growth factor receptor (FGFR) family, a receptor in the vascular endothelial growth factor receptor (VEGFR) family, a receptor in the rearranged during transfection (RET) receptor family, a receptor in the Eph receptor family, a receptor that can induce cell differentiation (e.g., a Notch receptor), a cell adhesion molecule (CAM), an adhesion receptor such as integrin receptor, cadherin, selectin, and a receptor in the discoidin domain receptor (DDR) family, transforming growth factor beta receptor 1, and transforming growth factor beta receptor 2. In some embodiments, such a receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

According to some embodiments, the protein of interest is an engineered cell surface receptor, and the engineered cell surface receptor is a chimeric antigen receptor (CAR). In certain embodiments, when the protein of interest is a CAR, the extracellular binding domain of the CAR comprises a single chain antibody. The single-chain antibody may be a monoclonal single-chain antibody, a chimeric single-chain antibody, a humanized single-chain antibody, a fully human single-chain antibody, and/or the like. In one non-limiting example, the single chain antibody is a single chain variable fragment (scFv). Suitable CAR extracellular binding domains include those described in Labanieh et al. (2018) Nature Biomedical Engineering 2:377-391. In some embodiments, the extracellular binding domain of the CAR is a single-chain version (e.g., an scFv version) of an antibody approved by the United States Food and Drug Administration and/or the European Medicines Agency (EMA) for use as a therapeutic antibody, e.g., for inducing antibody-dependent cellular cytotoxicity (ADCC) of certain disease-associated cells in a patient, etc. Non-limiting examples of single-chain antibodies which may be employed when the protein of interest is a CAR include single-chain versions (e.g., scFv versions) of Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, lmgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof. According to some embodiments, when the protein of interest is a CAR, the extracellular binding domain of the CAR specifically binds an antigen expressed on the surface of a cancer cell. For example, the extracellular binding domain may bind a cancer cell-surface antigen selected from B7-H3 (CD276), CD19, GD2, CD22, and HER2.

In certain embodiments, the protein of interest is a CAR includes one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a serine-glycine linker, such as a serine-glycine linker that includes the amino acid sequence GGGGSGGGGSGGGGS (G₄S)₃ (SEQ ID NO:26). In certain aspects, a linker separates one or more heavy or light chain variable domains, hinge domains, transmembrane domains, co-stimulatory domains, and/or primary signaling domains. In particular embodiments, the CAR includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In certain embodiments, the protein of interest is a CAR comprising an antigen binding domain followed by one or more spacer domains that moves the antigen binding domain away from the effector cell surface (e.g., the surface of a T cell expressing the CAR) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In one embodiment, the spacer domain includes the CH2 and/or CH3 of IgG1, IgG4, or IgD. Illustrative spacer domains suitable for use in the CARs described herein include the hinge region derived from the extracellular regions of type 1 membrane proteins such as CD8a and CD4, which may be wild-type hinge regions from these molecules or variants thereof. In certain aspects, the hinge domain includes a CD8a hinge region. In some embodiments, the hinge is a PD-1 hinge or CD152 hinge.

The “transmembrane domain” (TM domain) is the portion of the CAR that fuses the extracellular binding portion and intracellular signaling domain and anchors the CAR to the plasma membrane of the cell (e.g., immune effector cell). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cell receptor, CD35, CD3ζ, CD3γ, CD3δ, CD4, CD5, CD8α, CD9, CD16, CD22, CD27, CD28, CD33, CD37, CD45, CD64, CD80, CD86, CD134, CD137, CD152, CD154, and PD-1.

In certain embodiments, the protein of interest is a CAR that comprises a Tm domain derived from CD8a. According to some embodiments, a CAR includes a Tm domain derived from CD8a and a short oligo- or polypeptide linker, e.g., between 1, 2, 3, 4, 5, 6, 7, 8, 9, or 10 amino acids in length, that links the Tm domain and the intracellular signaling domain of the CAR. A glycine-serine linker may be employed as such a linker, for example.

The “intracellular signaling” domain of a CAR refers to the part of a CAR that participates in transducing the signal from CAR binding to a target molecule/antigen into the interior of the immune effector cell to elicit effector cell function, e.g., activation, cytokine production, proliferation and/or cytotoxic activity, including the release of cytotoxic factors to the CAR-bound target cell, or other cellular responses elicited with target molecule/antigen binding to the extracellular CAR domain. Accordingly, the term “intracellular signaling domain” refers to the portion(s) or domain(s) of a protein which transduce the effector function signal and that direct the cell to perform a specialized function. To the extent that a truncated portion of an intracellular signaling domain is used, such truncated portion may be used in place of a full-length intracellular signaling domain as long as it transduces the effector function signal. The term intracellular signaling domain is meant to include any truncated portion of an intracellular signaling domain sufficient for transducing effector function signal.

Signals generated through the T cell receptor (TCR) alone are insufficient for full activation of the T cell and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, when the protein of interest is a CAR, the CAR may include an intracellular signaling domain that includes one or more “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a CAR include those derived from FcRγ, FcRβ, CD3γ, CD3δ, CD3ε, CD3ζ, CD22, CD79α, CD79β, and CD66δ. In certain embodiments, a CAR includes a CD3ζ primary signaling domain and one or more costimulatory signaling domains. The intracellular primary signaling and costimulatory signaling domains are operably linked to the carboxyl terminus of the transmembrane domain.

In some embodiments, when the protein of interest is a CAR, the CAR includes one or more costimulatory signaling domains to enhance the efficacy and expansion of T cells expressing the CAR. As used herein, the term “costimulatory signaling domain” or “costimulatory domain” refers to an intracellular signaling domain of a costimulatory molecule or an active fragment thereof. Example costimulatory molecules suitable for use in CARs contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, a CAR includes one or more costimulatory signaling domains selected from the group consisting of 4-1BB, CD28, CD137, and CD134, and a CD3ζ primary signaling domain. In certain embodiments, the CAR comprises two or more intracellular signaling domains. For example, the CAR may comprise a first signaling domain and a second signaling domain or fragments thereof independently selected from a CD3ζ intracellular signaling domain, a CD28 intracellular signaling domain, a 4-1 BB intracellular signaling domain, an OX-40 intracellular signaling domain, an inducible co-stimulator (ICOS) intracellular signaling domain, a CD27 intracellular signaling domain, and a MyD88/CD40 intracellular signaling domain. By way of example, a CAR may include a first intracellular signaling domain or fragment thereof that is a CD3ζ intracellular signaling domain and a second intracellular signaling domain or fragment thereof that is a CD28 intracellular signaling domain. Also by way of example, a CAR may include a first intracellular signaling domain or fragment thereof that is a CD3ζ intracellular signaling domain and a second intracellular signaling domain or fragment thereof that is a 4-1 BB intracellular signaling domain.

According to some embodiments, when the protein of interest is a CAR, the CAR comprises an antigen-binding portion (e.g., a single chain antibody, such as an scFv) that binds to an antigen of interest; a transmembrane domain from a polypeptide selected from the group consisting of: CD4, CD8α, CD154, and PD-1; one or more intracellular costimulatory signaling domains from a polypeptide selected from the group consisting of: 4-1 BB, CD28, CD134, and CD137; and an intracellular signaling domain from a polypeptide selected from the group consisting of: FcRγ, FcRβ, CD3γ, CD3δ, CD3ε, CD3ζ, CD22, CD79α, CD79β, and CD66δ. Such a CAR may further include a spacer domain between the antigen-binding portion and the transmembrane domain, e.g., a CD8α hinge.

In certain embodiments, the protein of interest is a cell surface molecule, e.g., a cell surface receptor. According to some embodiments, the cell surface molecule is selected from a cytokine receptor, a chemokine receptor, an adhesion molecule, an integrin, an inhibitory receptor, an inhibitory cell surface ligand, a stimulatory receptor, a stimulatory cell surface ligand, an immunoreceptor tyrosine-based activation motif (ITAM)—containing receptor, and an immunoreceptor tyrosine-based inhibition motif (ITIM)—containing receptor.

When the protein of interest is a cell surface molecule (e.g., a cell surface receptor such as a CAR, an engineered or non-engineered TCR, etc.), the cell surface molecule may include an extracellular binding domain that specifically binds a molecule on the surface of a target cell. The target cell may be any cell type of interest. For example, the target cell may be a genetically and/or phenotypically normal cell. In other embodiments, the target cell is a genetically and/or phenotypically abnormal cell. Abnormal cells of interest include, but are not limited to, cancer cells, cells in the tumor microenvironment (e.g., tumor stromal cells) such as cancer-associated fibroblasts (CAFs), myeloid-derived suppressor cells (MDSCs), tumor-associated macrophages (TAMs), tumor endothelial cells (TECs), and the like. See, e.g., Labanieh et al. (2018) Nature Biomedical Engineering 2:377-391. By “cancer cell” is meant a cell exhibiting a neoplastic cellular phenotype, which may be characterized by one or more of the following exemplary characteristics: abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like. According to some embodiments, the protein of interest is a TCR that recognizes an antigenic peptide complexed with a major histocompatibility complex (MHC) molecule displayed on the surface of a cancer cell.

In certain embodiments, when the target cell is a cancer cell, the cell surface molecule (e.g., CAR, TCR, or the like) specifically binds to a tumor antigen on the surface of the cancer cell. Non-limiting examples of tumor antigens to which the cell surface molecule may specifically bind include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), trophoblast cell-surface antigen (TROP-2), and Wilms' tumor 1 (WT1).

When the protein of interest is a cell surface molecule (e.g., a cell surface receptor such as a CAR, an engineered or non-engineered TCR, etc.), the cell surface molecule may include an extracellular binding domain that specifically binds to an antigen, e.g., a cell surface antigen, such as an antigen on the surface of a cancer cell, or an antigenic peptide associated with an MHC molecule. The extracellular binding domain “specifically binds” to the antigen if it binds to or associates with the antigen with an affinity or Kₐ (that is, an equilibrium association constant of a particular binding interaction with units of 1/M) of, for example, greater than or equal to about 10⁵ M⁻¹. In certain embodiments, the extracellular binding domain binds to an antigen with a K a greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a K a of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the extracellular binding domain binds to the target molecule with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the extracellular binding domain for the target antigen can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

According to some embodiments, the protein of interest component of a recombinant polypeptide of the present disclosure is a transcription factor. Such recombinant polypeptides find use, e.g., when it is desirable to regulate the expression of target genes of the transcription factor by regulating the ability of the transcription factor to localize to the nucleus. For example, the protein localization tag may cause retention of the transcription factor at a compartment of the cell other than the nucleus when an inhibitor of the protease is present, whereas the transcription factor localizes to the nucleus in the absence of the protease inhibitor.

In certain embodiments, the protein of interest of a recombinant polypeptide of the present disclosure is a secreted effector molecule. By “secreted effector molecule” is meant an effector molecule (e.g., a stimulatory ligand, an inhibitory ligand, a cytokine, a chemokine, a growth factor, a protease, or the like) that is secreted by the cell when the protein localization tag is cleaved from the effector molecule. According to some embodiments, when the protein of interest is a secreted effector molecule, the protein localization tag is an ER localization tag. Non-limiting example configurations of recombinant polypeptides comprising a secreted effector molecule and ER localization tag are schematically illustrated in FIG. 9.

Proteases and Protease Cleavage Sites

The recombinant polypeptides comprise a protease cleavage site disposed between the protein of interest and the protein localization tag. The term “cleavage site” refers to the bond (e.g., a scissile bond) cleaved by an agent, e.g., a protease. A cleavage site for a protease includes the specific amino acid sequence recognized by the protease during proteolytic cleavage and may include surrounding amino acids (e.g., from one to six amino acids) on either side of the scissile bond, which bind to the active site of the protease and are needed for recognition as a substrate. In some embodiments, the cleavage site is provided as a cleavable linker, where “cleavable linker” refers to a linker including the protease cleavage site. A cleavable linker is typically cleavable under physiological conditions.

In certain embodiments, the polypeptides further comprise a protease (referred to herein as a “cis” configuration), where the protease cleavage site is a cleavage site for the protease. According to some embodiments, when a recombinant polypeptide comprises the protease, the polypeptide comprises from N-terminus to C-terminus: the protein of interest; the protease cleavage site; the protease; and the protein localization tag. In certain embodiments, when a recombinant polypeptide comprises the protease, the polypeptide comprises from N-terminus to C-terminus: the protein localization tag; the protease; the protease cleavage site; and the protein of interest. In certain embodiments, the polypeptides do not comprise a protease that cleaves the protease cleavage site, but such a protease capable of cleaving the protease cleavage site is expressed as a separate molecule—referred to herein as a “trans” configuration. Non-limiting examples of cis and trans configurations are schematically illustrated in FIGS. 8 and 9.

In some embodiments, the protease is highly selective for the cleavage site in the cell surface receptor. Additionally, protease activity is preferably capable of inhibition by known small molecule inhibitors that are cell-permeable and not toxic to the cell or individual under study or treatment. For a discussion of proteases, see, e.g., V. Y. H. Hook, Proteolytic and cellular mechanisms in prohormone and proprotein processing, RG Landes Company, Austin, Tex., USA (1998); N. M. Hooper et al., Biochem. J. 321: 265-279 (1997); Z. Werb, Cell 91: 439-442 (1997); T. G. Wolfsberg et al., J. Cell Biol. 131: 275-278 (1995); T. Berg et al., Biochem. J. 307: 313-326 (1995); M. J. Smyth and J. A. Trapani, Immunology Today 16: 202-206 (1995); R. V. Talanian et al., J. Biol. Chem. 272: 9677-9682 (1997); and N. A. Thornberry et al., J. Biol. Chem. 272: 17907-17911 (1997), the disclosures of which are incorporated herein by reference in their entireties for all purposes.

In some embodiments, the protease employed is a sequence-specific non-human protease for which FDA-approved pharmacological inhibitors are available. In some embodiments, the protease employed is a viral protease. Non-limiting example viral proteases that may be used with the systems, compositions, and methods provided herein include a hepatitis C virus (HCV) protease, a rhinovirus protease, a coxsackie virus protease, a dengue virus protease, and a tev protease. According to some embodiments, the viral protease is an HCV protease. In certain embodiments, the viral protease is derived from HCV nonstructural protein 3 (NS3). NS3 consists of an N-terminal serine protease domain and a C-terminal helicase domain. By “derived from HCV NS3” is meant the protease is the serine protease domain of HCV NS3 or a proteolytically active variant thereof capable of cleaving a cleavage site for the serine protease domain of HCV NS3. The protease domain of NS3 forms a heterodimer with the HCV nonstructural protein 4A (NS4A), which activates proteolytic activity. A protease derived from HCV NS3 may include the entire NS3 protein or a proteolytically active fragment thereof, and may further include a cofactor polypeptide, such as a cofactor polypeptide derived from HCV nonstructural protein 4A (NS4A), e.g., an activating NS4A region. NS3 protease is highly selective and can be inhibited by a number of non-toxic, cell-permeable drugs, which are currently available for use in humans. NS3 protease inhibitors that may be employed include, but are not limited to, simeprevir, danoprevir, asunaprevir, ciluprevir, boceprevir, sovaprevir, paritaprevir, telaprevir, grazoprevir, and any combination thereof. Non-limiting examples of proteases derived from HCV NS3 are provided below.

Example Proteases Derived from HCV NS3
(SEQ ID NO: 27)
APITAYAQQTRGLLGCIITSLTGRDKNQVEGEVQIVSTATQTFLATCING
VCWAVYHGAGTRTIASPKGPVIQMYTNVDQDLVGWPAPQGSRSLTPCTCG
SSDLYLVTRHADVIPVRRRGDSRGSLLSPRPISYLKGSSGGPLLCPAGHA
VGLFRAAVCTRGVAKAVDFIPVENLETTMRSPVFTD
(SEQ ID NO: 28)
APITAYAQQTRGLLGCIITSLTGRDKNQVEGEVQIMSTATQTFLATCING
VCWTVYHGAGTRTIASPKGPVIQMYTNVDQDLVGWPAPQGSRSLTPCTCG
SSDLYLVTRHADVIPVRRRGDGRGSLLSPRPISYLKGSSGGPLLCPAGHA
VGLFRAAVCTRGVAKAVDFIPVENLETTMRSPVFTD
(SEQ ID NO: 29)
APITAYAQQTRGLLGCIITSLTGRDKNQVEGEVQIVSTATQTFLATCING
VCWAVYHGAGTRTIASPKGPVIQMYTNVDQDLVGWPAPQGSRSLTPCTCG
SSDLYLVTRHADVIPVRRRGDSRGSLLSPRPISYLKGSSGGPLLCPAGHA
VGLFRAAVCTRGVAKAVDFIPVENLETTMRSPVFTD
(SEQ ID NO: 30)
APITAYAQQTRGLLGCIITSLTGRDKNQVEGEVQIVSTATQTFLATCING
VCWTVYHGAGTRTIASPKGPVIQMYTNVDQDLVGWPAPQGSRSLTPCTCG
SSDLYLVTRHADVIPVRRRGDSRGSLLSPRPISYLKGSSGGPLLCPAGHA
VGLFRAAVCTRGVAKAVDFIPVENLETTMRSPVFTD

In some embodiments, the protease includes the sequence set forth in SEQ ID NO: 27, SEQ ID NO:28, SEQ ID NO:29, or SEQ ID NO:30, or is a functional (proteolytic) variant thereof having 70% or greater, 75% or greater, 80% or greater, 85% or greater, 90% or greater, 95% or greater, or 99% or greater amino acid sequence identity to SEQ ID NO:27, SEQ ID NO:28, SEQ ID NO:29, or SEQ ID NO:30, and/or a functional (proteolytic) fragment thereof such as a fragment having a length of from 100 to 185, 120 to 185, 140 to 185, 160 to 185, 170 to 185, from 180 to 185, from 182 to 185, or from 184 to 185 amino acids.

In some embodiments, the protease cleavage site is a viral protease cleavage site. For example, when a protease derived from HCV NS3 is employed, the cleavage site should comprise an NS3 protease cleavage site. An NS3 protease cleavage site may include the four junctions between nonstructural (NS) proteins of the HCV polyprotein normally cleaved by the NS3 protease during HCV infection, including the NS3/NS4A, NS4A/NS4B, NS4B/NSSA, and NSSA/NSSB junction cleavage sites. For a description of NS3 protease and representative sequences of its cleavage sites for various strains of HCV, see, e.g., Hepatitis C Viruses: Genomes and Molecular Biology (S. L. Tan ed., Taylor & Francis, 2006), Chapter 6, pp. 163-206; the disclosure of which is incorporated herein by reference in its entirety.

In some embodiments, the protease is derived from HCV NS3 and engineered to include one or more amino acid substitutions relative to the amino acid sequence set forth in SEQ ID NO:27. For example, the protease may include a substitution at the position corresponding to position 54 of the amino acid sequence set forth in SEQ ID NO:27. In some embodiments, such a substitution is a threonine to alanine substitution.

NS3 nucleic acid and protein sequences may be derived from HCV, including any isolate of HCV having any genotype (e.g., genotypes 1-7) or subtype. A number of NS3 nucleic acid and protein sequences are known and described, e.g., in U.S. Ser. No. 15/737,712, the disclosure of which is incorporated herein by reference in their entirety for all purposes. Additional representative NS3 sequences are listed in the National Center for Biotechnology Information (NCBI) database. See, for example, NCBI entries: Accession Nos. YP_001491553, YP_001469631, YP_001469632, NP_803144, NP_671491, YP_001469634, YP_001469630, YP_001469633, ADA68311, ADA68307, AFP99000, AFP98987, ADA68322, AFP99033, ADA68330, AFP99056, AFP99041, CBF60982, CBF60817, AHH29575, A1Z00747, A1Z00744, ABI36969, ABN05226, KF516075, KF516074, KF516056, AB826684, AB826683, JX171009, JX171008, JX171000, EU847455, EF154714, GU085487, JX171065, and JX171063; all of which sequences are herein incorporated by reference. Any of these sequences or functional variants thereof having 70% or greater, 75% or greater, 80% or greater, 85% or greater, 90% or greater, 95% or greater, or 99% or greater amino acid sequence identity to any one of these sequences, or proteolytic fragments thereof, may be employed.

NS4A nucleic acid and protein sequences may be derived from HCV, including any isolate of HCV having any genotype (e.g., seven genotypes 1-7) or subtype. A number of NS4A nucleic acid and protein sequences are known. Representative NS4A sequences are listed in the National Center for Biotechnology Information (NCBI) database. See, for example, NCBI entries: Accession Nos. NP_751925, YP_001491554, GU945462, HQ822054, FJ932208, FJ932207, FJ932205, and FJ932199; all of which sequences (as entered by the date of filing of this application) are herein incorporated by reference. Any of these sequences or functional variants thereof having 70% or greater, 75% or greater, 80% or greater, 85% or greater, 90% or greater, 95% or greater, or 99% or greater amino acid sequence identity to any one of these sequences, or proteolytic fragments thereof, may be employed.

HCV polyprotein nucleic acid and protein sequences may be derived from HCV, including any isolate of HCV having any genotype (e.g., genotypes 1-7) or subtype. A number of HCV polyprotein nucleic acid and protein sequences are known. Representative HCV polyprotein sequences are listed in the National Center for Biotechnology Information (NCBI) database. See, for example, NCBI entries: Accession Nos. YP_001469631, NP_671491, YP_001469633, YP_001469630, YP_001469634, YP_001469632, NC_009824, NC_004102, NC_009825, NC_009827, NC_009823, NC_009826, and EF108306; all of which sequences (as entered by the date of filing of this application) are herein incorporated by reference. Any of these sequences or functional variants thereof having 70% or greater, 75% or greater, 80% or greater, 85% or greater, 90% or greater, 95% or greater, or 99% or greater amino acid sequence identity to any one of these sequences, or proteolytic fragments thereof, may be employed.

In some embodiments, the protease is derived from HCV NS3 and the cleavage site includes an NS3 protease cleavage site. An NS3 protease cleavage site may include the HCV polyprotein NS3/NS4A, NS4A/NS4B, NS4B/NSSA, and NSSA/NSSB junction cleavage sites. Representative HCV NS4A/4B protease cleavage sites include DEMEECSQH (SEQ ID NO:31) and DEMEECSQH (SEQ ID NO:32). Representative HCV NSSA/5B protease cleavage sites include EDVVPCSMG (SEQ ID NO:33) and EDVVPCSMGS (SEQ ID NO:34). A representative NS4B/5A protease cleavage site is ECTTPCSGSWL (SEQ ID NO:35). Additional NS3 protease cleavage sites that may be included in a recombinant polypeptide of the present disclosure include those described in Shiryaev et al. (2012) PLoS One 7(4):e35759.

### Nucleic Acids

Also provided by the present disclosure are nucleic acids encoding any of the recombinant polypeptides and/or proteases (e.g., extracellularly- or intracellularly-tethered protease constructs) of the present disclosure, including any of the recombinant polypeptides and/or proteases having any of the features (e.g., domains, etc.) and combinations thereof described hereinabove. Because the genetic code is degenerate, there are many nucleotide sequences that may encode the recombinant polypeptides and/or proteases of the present disclosure. Some of these polynucleotides may bear minimal homology to the nucleotide sequence of any native gene. Polynucleotides that vary due to differences in codon usage are specifically contemplated in particular embodiments, for example polynucleotides that are optimized for human and/or primate codon selection.

Shown in Table 1 below are amino acid sequences of example recombinant polypeptides and proteases of the present disclosure (shown from N- to C-terminus). Included in these examples are the recombinant polypeptides employed in the Experimental section below. Not shown are signal sequences initially present at the N-termini of the polypeptides. Segments/domains of the polypeptides are indicated by alternating stretches of underlined and non-underlined text, and the identities of the segments/domains are provided in the left column.

TABLE 1
Example Recombinant Polypeptides and Protease Sequences

| | |
|---|---|
| B7H3 CAR-STASH ER 1 | EVQLVESGGGLVQPGGSLRLSCAASGFTFSSFGMHWVRQA |
| (SEQ ID NO: 36) | PGKGLEWVAYISSDSSAIYYADTVKGRFTISRDNAKNSLYLQ |
| B7H3 scFv (MGA271) heavy | MNSLRDEDTAVYYCGRGRENIYYGSRLDYWGQGTTVTVSS |
| chain | GGGGSGGGGSGGGGSDIQLTQSPSFLSASVGDRVTITCKAS |
| Linker | QNVDTNVAWYQQKPGKAPKALIYSASYRYSGVPSRFSGSGS |
| B7H3 scFv (MGA271) light chain | GTDFTLTISSLQPEDFATYYCQQYNNYPFTFGQGTKLEIKAAA |
| Linker | TTTPAPRPPTPAPTIASQPLSLRPEACRPAAGGAVHTRGLDF |
| CD8 alpha hinge | ACDIYIWAPLAGTCGVLLLSLVITLYCKRGRKKLLYIFKQPFMR |
| CD8 alpha transmembrane | PVQTTQEEDGCSCRFPEEEEGGCELRVKFSRSADAPAYKQ |
| domain | GQNQLYNELNLGRREEYDVLDKRRGRDPEMGGKPRRKNPQ |
| 4-1BB costimulatory domain | EGLYNELQKDKMAEAYSEIGMKGERRRGKGHDGLYQGLST |
| CD3 zeta intracellular domain | ATKDTYDALHMQALPPRSIGGGSGGSDEMEECSQHGGSGG |
| Linker | STGCVVIVGRIVLSGSGTSAPITAYAQQTRGLLGCIITSLTGRD |
| NS4a4b cleavage site | KNQVEGEVQIMSTATQTFLATCINGVCWAVYHGAGTRTIASP |
| Linker | KGPVIQMYTNVDQDLVGWPAPQGSRSLTPCTCGSSDLYLVT |
| NS4A cofactor domain | RHADVIPVRRRGDGRGSLLSPRPISYLKGSSGGPLLCPAGHA |
| Linker | VGLFRAAVCTRGVAKAVDFIPVENLETTMRSPVFTDNSSPPA |
| NS3 protease | VTLTHGGSGGSLYKYKSRRSFIDEKKMP |
| NS3 helicase fragment | |
| Linker | |
| ER localization tag | |
| B7H3 CAR-STASH ER 2 | EVQLVESGGGLVQPGGSLRLSCAASGFTFSSFGMHWVRQA |
| (SEQ ID NO: 37) | PGKGLEWVAYISSDSSAIYYADTVKGRFTISRDNAKNSLYLQ |
| B7H3 scFv (MGA271) heavy | MNSLRDEDTAVYYCGRGRENIYYGSRLDYWGQGTTVTVSS |
| chain | GGGGSGGGGSGGGGSDIQLTQSPSFLSASVGDRVTITCKAS |
| linker | QNVDTNVAWYQQKPGKAPKALIYSASYRYSGVPSRFSGSGS |
| B7H3 scFv (MGA271) light chain | GTDFTLTISSLQPEDFATYYCQQYNNYPFTFGQGTKLEIKAAA |
| linker | TTTPAPRPPTPAPTIASQPLSLRPEACRPAAGGAVHTRGLDF |
| CD8 alpha hinge | ACDIYIWAPLAGTCGVLLLSLVITLYCKRGRKKLLYIFKQPFMR |
| CD8 alpha transmembrane | PVQTTQEEDGCSCRFPEEEEGGCELRVKFSRSADAPAYKQ |
| domain | GQNQLYNELNLGRREEYDVLDKRRGRDPEMGGKPRRKNPQ |
| 4-1BB costimulatory domain | EGLYNELQKDKMAEAYSEIGMKGERRRGKGHDGLYQGLST |
| CD3 zeta domain | ATKDTYDALHMQALPPRSIGGGSGGSDEMEECSQHGGSGG |
| Linker | STGCVVIVGRIVLSGSGTSAPITAYAQQTRGLLGCIITSLTGRD |
| NS4a4b cleavage site | KNQVEGEVQIMSTATQTFLATCINGVCWAVYHGAGTRTIASP |
| Linker | KGPVIQMYTNVDQDLVGWPAPQGSRSLTPCTCGSSDLYLVT |
| NS4A cofactor domain | RHADVIPVRRRGDGRGSLLSPRPISYLKGSSGGPLLCPAGHA |
| Linker | VGLFRAAVCTRGVAKAVDFIPVENLETTMRSPVFTDNSSPPA |
| NS3 protease | VTLTHGGSGGSKKMP |
| NS3 helicase fragment | |
| Linker | |
| ER localization tag | |
| B7H3 CAR-STASH Golgi | EVQLVESGGGLVQPGGSLRLSCAASGFTFSSFGMHWVRQA |
| (SEQ ID NO: 38) | PGKGLEWVAYISSDSSAIYYADTVKGRFTISRDNAKNSLYLQ |
| B7H3 scFv (MGA271) heavy | MNSLRDEDTAVYYCGRGRENIYYGSRLDYWGQGTTVTVSS |
| chain | GGGGSGGGGSGGGGSDIQLTQSPSFLSASVGDRVTITCKAS |
| linker | QNVDTNVAWYQQKPGKAPKALIYSASYRYSGVPSRFSGSGS |
| B7H3 scFv (MGA271) light chain | GTDFTLTISSLOPEDFATYYCQQYNNYPFTFGQGTKLEIKAAA |
| linker | TTTPAPRPPTPAPTIASQPLSLRPEACRPAAGGAVHTRGLDF |
| CD8 alpha hinge | ACDIYIWAPLAGTCGVLLLSLVITLYCKRGRKKLLYIFKQPFMR |
| CD8 alpha transmembrane | PVQTTQEEDGCSCRFPEEEEGGCELRVKFSRSADAPAYKQ |
| domain | GQNQLYNELNLGRREEYDVLDKRRGRDPEMGGKPRRKNPQ |
| 4-1BB costimulatory domain | EGLYNELQKDKMAEAYSEIGMKGERRRGKGHDGLYQGLST |
| CD3 zeta domain | ATKDTYDALHMQALPPRSIGGGSGGSDEMEECSQHGGSGG |
| Linker | STGCVVIVGRIVLSGSGTSAPITAYAQQTRGLLGCIITSLTGRD |
| NS4a4b cleavage site | KNQVEGEVQIMSTATQTFLATCINGVCWAVYHGAGTRTIASP |
| Linker | KGPVIQMYTNVDQDLVGWPAPQGSRSLTPCTCGSSDLYLVT |
| NS4A cofactor domain | RHADVIPVRRRGDGRGSLLSPRPISYLKGSSGGPLLCPAGHA |
| Linker | VGLFRAAVCTRGVAKAVDFIPVENLETTMRSPVFTDNSSPPA |
| NS3 protease | VTLTHGGSGGSYQRL |
| NS3 helicase fragment | |
| Linker | |
| Golgi localization tag | |
| B7H3 CAR-STASH Lysosome | EVQLVESGGGLVQPGGSLRLSCAASGFTFSSFGMHWVRQA |
| (SEQ ID NO: 39) | PGKGLEWVAYISSDSSAIYYADTVKGRFTISRDNAKNSLYLQ |
| B7H3 scFv (MGA271) heavy | MNSLRDEDTAVYYCGRGRENIYYGSRLDYWGQGTTVTVSS |
| chain | GGGGSGGGGSGGGGSDIQLTQSPSFLSASVGDRVTITCKAS |
| linker | QNVDTNVAWYQQKPGKAPKALIYSASYRYSGVPSRFSGSGS |
| B7H3 scFv (MGA271) light chain | GTDFTLTISSLQPEDFATYYCQQYNNYPFTFGQGTKLEIKAAA |
| linker | TTTPAPRPPTPAPTIASQPLSLRPEACRPAAGGAVHTRGLDF |
| CD8 alpha hinge | ACDIYIWAPLAGTCGVLLLSLVITLYCKRGRKKLLYIFKQPFMR |
| CD8 alpha transmembrane | PVQTTQEEDGCSCRFPEEEEGGCELRVKFSRSADAPAYKQ |
| domain | GQNQLYNELNLGRREEYDVLDKRRGRDPEMGGKPRRKNPQ |
| 4-1BB costimulatory domain | EGLYNELQKDKMAEAYSEIGMKGERRRGKGHDGLYQGLST |
| CD3 zeta domain | ATKDTYDALHMQALPPRSIGGGSGGSDEMEECSQHGGSGG |
| Linker | STGCVVIVGRIVLSGSGTSAPITAYAQQTRGLLGCIITSLTGRD |
| NS4a4b cleavage site | KNQVEGEVQIMSTATQTFLATCINGVCWAVYHGAGTRTIASP |
| Linker | KGPVIQMYTNVDQDLVGWPAPQGSRSLTPCTCGSSDLYLVT |
| NS4A cofactor domain | RHADVIPVRRRGDGRGSLLSPRPISYLKGSSGGPLLCPAGHA |
| Linker | VGLFRAAVCTRGVAKAVDFIPVENLETTMRSPVFTDNSSPPA |
| NS3 protease | VTLTHGGSGGSKFERQ |
| NS3 helicase fragment | |
| Linker | |
| Lysosome localization tag | |
| HCV NS3 Protease (Intracellularly | ACPYSNPSLCSGGGGSELPTQGTFSNVSTNVSPAKPTTTAC |
| Tethered) amino acid sequence | PYSNPSLCSGGGGSPAPRPPTPAPTIASQPLSLRPEACRPAA |
| (SEQ ID NO: 40) | GGAVHTRGLDFACDIYIWAPLAGTCGVLLLSLVITLYCNHRNR |
| CD20 epitope | RRVCKCPRPVVGSSGNSSGGSTGCVVIVGRIVLSGSGTSAPI |
| linker | TAYAQQTRGLLGCIITSLTGRDKNQVEGEVQIMSTATQTFLAT |
| CD34 epitope | CINGVCWAVYHGAGTRTIASPKGPVIQMYTNVDQDLVGWPA |
| CD8 alpha peptide fragment | PQGSRSLTPCTCGSSDLYLVTRHADVIPVRRRGDGRGSLLS |
| CD20 alpha stalk | PRPISYLKGSSGGPLLCPAGHAVGLFRAAVCTRGVAKAVDFI |
| CD8 alpha transmembrane | PVENLETTMRSPVFTDNSSPPAVTLTH |
| CD8 alpha intracellular fragment | |
| linker | |
| NS4a cofactor domain | |
| linker | |
| NS3 protease | |
| NS3 helicase fragment | |

Also provided are expression vectors comprising any of the nucleic acids of the present disclosure. A “vector” is a nucleic acid molecule capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

In order to express a desired recombinant polypeptide and/or protease, a nucleotide sequence encoding the recombinant polypeptide and/or protease can be inserted into an appropriate vector, e.g., using recombinant DNA techniques known in the art. Exemplary viral vectors include, without limitation, retrovirus (including lentivirus), adenovirus, adeno-associated virus, herpesvirus (e.g., herpes simplex virus), poxvirus, papillomavirus, and papovavirus (e.g., SV40). Illustrative examples of expression vectors include, but are not limited to, pClneo vectors (Promega) for expression in mammalian cells; pLenti4/V 5-DEST™, pLenti6/V 5-DEST™, murine stem cell virus (MSCV), MSGV, moloney murine leukemia virus (MMLV), and pLenti6.2/V5-GW/lacZ (Invitrogen) for lentivirus-mediated gene transfer and expression in mammalian cells. In certain embodiments, a nucleic acid sequence encoding a recombinant polypeptide and/or protease of the present disclosure may be ligated into any such expression vectors for the expression of the recombinant polypeptide and/or protease in mammalian cells.

In some embodiments, when the recombinant polypeptide and the protease are employed in trans, the recombinant polypeptide and the protease are expressed from separate expression vectors. In some embodiments, when the recombinant polypeptide and the protease are employed in trans, the cell surface receptor and the protease are expressed from the same expression vector. In some embodiments, such an expression vector is a bicistronic expression vector where the recombinant polypeptide and the protease are expressed under the same promoter. For example, the expression vector may include an internal ribosome entry site (IRES) or a ribosome skipping site (sometimes referred to as a self-cleaving peptide sequence) such as a porcine teschovirus-1 2A (P2A) sequence, Thosea asigna virus 2A (T2A) sequence, foot-and-mouth disease virus 2A (F2A) sequence, and equine rhinitis A virus 2A (E2A) sequence between the recombinant polypeptide- and protease-encoding regions, permitting the recombinant polypeptide and the protease to be expressed as separate polypeptides from the same promoter. Further details regarding ribosome skipping sites for use in polycistronic vectors may be found, e.g., in Liu et al. (2017) Scientific Reports 7:2193.

Expression control sequences, control elements, or regulatory sequences present in an expression vector are those non-translated regions of the vector—e.g., origins of replication, selection cassettes, promoters, enhancers, translation initiation signals (Shine Dalgarno sequence or Kozak sequence), introns, a polyadenylation sequence, 5′ and 3′ untranslated regions, and/or the like—which interact with host cellular proteins to carry out transcription and translation. Such elements may vary in their strength and specificity, and can be selected by one skilled in the art depending on the vector system and host to be used for each particular construct. Depending on the vector system and host utilized, any number of suitable transcription and translation elements, including ubiquitous promoters and inducible promoters may be used.

Components of the expression vector are operably linked such that they are in a relationship permitting them to function in their intended manner. In some embodiments, the term refers to a functional linkage between a nucleic acid expression control sequence (such as a promoter, and/or enhancer) and a second polynucleotide sequence, e.g., a nucleic acid encoding the recombinant polypeptide and/or the protease, where the expression control sequence directs transcription of the nucleic acid encoding the recombinant polypeptide and/or the protease.

In some embodiments, the expression vector is an episomal vector or a vector that is maintained extrachromosomally. As used herein, the term “episomal” refers to a vector that is able to replicate without integration into the host cell's chromosomal DNA and without gradual loss from a dividing host cell also meaning that the vector replicates extrachromosomally or episomally. Such a vector may be engineered to harbor the sequence coding for the origin of DNA replication or “ori” from an alpha, beta, or gamma herpesvirus, an adenovirus, SV40, a bovine papilloma virus, a yeast, or the like. The host cell may include a viral replication transactivator protein that activates the replication. Alpha herpes viruses have a relatively short reproductive cycle, variable host range, efficiently destroy infected cells and establish latent infections primarily in sensory ganglia. Illustrative examples of alpha herpes viruses include HSV 1, HSV 2, and VZV. Beta herpesviruses have long reproductive cycles and a restricted host range. Infected cells often enlarge. Non-limiting examples of beta herpes viruses include CMV, HHV-6 and HHV-7. Gamma-herpesviruses are specific for either T or B lymphocytes, and latency is often demonstrated in lymphoid tissue. Illustrative examples of gamma herpes viruses include EBV and HHV-8.

Other gene delivery systems which may be used include mRNA electroporation, CRISPR-Cas9, TALENs, zinc fingers, transposase vectors, and the like. See, e.g., Labanieh et al. (2018) Nature Biomedical Engineering 2:377-391.

### Cells

Also provided are cells (e.g., recombinant host cells) comprising any of the recombinant polypeptides, proteases, nucleic acids, and/or expression vectors of the present disclosure.

In some embodiments, the cells are eukaryotic cells. Eukaryotic cells of interest include, but are not limited to, yeast cells, insect cells, mammalian cells, and the like. Mammalian cells of interest include, e.g., murine cells, non-human primate cells, human cells, and the like.

The terms “recombinant host cells,” “host cells,” “cells,” “cell lines,” “cell cultures,” and other such terms refer to cells which can be, or have been, used as recipients for a recombinant vector or other transferred DNA, and include the progeny of the cell which has been transfected. Host cells may be cultured as unicellular or multicellular entities (e.g., tissue, organs, or organoids) including an expression vector of the present disclosure.

In one aspect, the cells provided herein include immune cells. Non-limiting examples of immune cells which may include any of the recombinant polypeptides, proteases, nucleic acids, and/or expression vectors of the present disclosure include T cells, B cells, natural killer (NK) cells, a macrophages, monocytes, neutrophils, dendritic cells, mast cells, basophils, and eosinophils. In some embodiments, the immune cell comprises a T cell. Exemplary T cell types include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells (T_{H}, T_{H}1, T_{H}2, T_{H}17) CD4+ T cells, CD8+ T cells, virus-specific T cells, alpha beta T cells (T_{αβ}), and gamma delta T cells (T_{γδ}). In some embodiments, the cell is a T cell and the protein of interest is a CAR, e.g., any of the CARs described herein. In another aspect, the cells provided herein comprise stem cells, e.g., an embryonic stem cell or an adult stem cell.

In one aspect, the cells provided herein comprise stem cells and progenitor cells. Non-limiting examples of stem cells which may include any of the recombinant polypeptides, proteases, nucleic acids, and/or expression vectors of the present disclosure include hematopoietic stem cells (HSCs), induced pluripotent stem cells (iPSCs), mesenchymal stem cells (MSCs), and neural stem cells (NSCs).

When a cell of the present disclosure includes a protease capable of cleaving the protease cleavage site in the absence of an inhibitor, the protease may be a soluble cytosolic protease (that is—not associated/tethered to a membrane), or the protease may be tethered intracellularly or extracellularly to the cell membrane. In some embodiments, when a cell of the present disclosure includes the protease, the recombinant polypeptide comprises the protease.

Also provided are methods of making the cells of the present disclosure. In some embodiments, such methods include transfecting or transducing cells with a nucleic acid or expression vector of the present disclosure. The term “transfection” or “transduction” is used to refer to the introduction of foreign DNA into a cell. A cell has been “transfected” when exogenous DNA has been introduced inside the cell membrane. A number of transfection techniques are generally known in the art. See, e.g., Sambrook et al. (2001) Molecular Cloning, a laboratory manual, 3 rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material.

In some embodiments, a cell of the present disclosure is produced by transfecting the cell with a viral vector encoding the recombinant polypeptide. In some embodiments, the protein of interest of the recombinant polypeptide is a CAR and the cell is a T cell, such that provided are methods of producing a CAR T cell in which cell surface expression of the CAR is regulatable. By “cell surface expression” or “expressed on the surface of the cell” is meant the cell surface molecule—when no longer associated with the protein localization tag (e.g., ER localization tag, Golgi localization tag, or the like) has been trafficked to the cell membrane such that—in the case of a cell surface receptor (e.g., a CAR, TCR, etc.)—the extracellular binding domain is displayed on the cell surface, the transmembrane portion passes through the cell membrane, and the one or more intracellular signaling domains are disposed adjacent to the intracellular side of the cell membrane. Upon binding of the extracellular binding domain to the target ligand/antigen, the intracellular signaling domain of the cell surface receptor participates in transducing the signal from the binding into the interior of the cell (e.g., an effector cell, such as a T cell, to elicit effector cell function).

In some embodiments, when the protein of interest is a CAR, the methods of producing a CAR T cell include activating a population of T cells (e.g., T cells obtained from an individual to whom a CAR T cell therapy will be administered), stimulating the population of T cells to proliferate, and transducing the T cell with a viral vector encoding the CAR. In certain embodiments, the T cells are transduced with a retroviral vector, e.g., a gamma retroviral vector or a lentiviral vector, encoding the CAR. In some embodiments, the T cells are transduced with a lentiviral vector encoding the CAR.

Cells of the present disclosure may be autologous/autogeneic (“self”) or non-autologous (“non-self,” e.g., allogeneic, syngeneic or xenogeneic). “Autologous” as used herein, refers to cells derived from the same individual to which they are subsequently administered. “Allogeneic” as used herein refers to cells of the same species that differ genetically from the cell in comparison. “Syngeneic,” as used herein, refers to cells of a different individual that are genetically identical to the cell in comparison. In some embodiments, the cells are T cells obtained from a mammal. In some embodiments, the mammal is a primate. In some embodiments, the primate is a human.

T cells may be obtained from a number of sources including, but not limited to, peripheral blood, peripheral blood mononuclear cells, bone marrow, lymph node tissue, cord blood, thymus tissue, tissue from a site of infection, ascites, pleural effusion, spleen tissue, and tumors. In certain embodiments, T cells can be obtained from a unit of blood collected from an individual using any number of known techniques such as sedimentation, e.g., FICOLL™ separation.

In some embodiments, an isolated or purified population of T cells is used. In some embodiments, T_{CTL} and T_{H} lymphocytes are purified from PBMCs. In some embodiments, the T_{CTL} and T_{H} lymphocytes are sorted into naïve (T_{N}), memory (T_{HEM}), stem cell memory (T_{SCM}), central memory (T_{CM}), effector memory (T_{EM}), and effector (T_{EFF}) T cell subpopulations either before or after activation, expansion, and/or genetic modification. Suitable approaches for such sorting are known and include, e.g., magnetic-activated cell sorting (MACS), where TN are CD45RA⁺ CD62L⁺ CD95⁻; TSCM are CD45RA⁺ CD62L⁺ CD95⁺; TCM are CD45RO⁺ CD62L⁺ CD95⁺; and TEM are CD45RO⁺ CD62L⁻ CD95⁺. An exemplary approach for such sorting is described in Wang et al. (2016) Blood 127(24):2980-90.

A specific subpopulation of T cells expressing one or more of the following markers: CD3, CD4, CD8, CD28, CD45RA, CD45RO, CD62, CD127, and HLA-DR can be further isolated by positive or negative selection techniques. In some embodiments, a specific subpopulation of T cells, expressing one or more of the markers selected from the group consisting of CD62L, CCR7, CD28, CD27, CD122, CD127, CD197; or CD38 or CD62L, CD127, CD197, and CD38, is further isolated by positive or negative selection techniques. In some embodiments, the manufactured T cell compositions do not express one or more of the following markers: CD57, CD244, CD 160, PD-1, CTLA4, TIM3, and LAG3. In some embodiments, the manufactured T cell compositions do not substantially express one or more of the following markers: CD57, CD244, CD 160, PD-1, CTLA4, TIM3, and LAG3.

In order to achieve therapeutically effective doses of T cell compositions, the T cells may be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes. In some embodiments, T cells are activated and expanded for about 1 to 21 days, e.g., about 5 to 21 days. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of a nucleic acid (e.g., expression vector) encoding the polypeptide into the T cells.

In some embodiments, T cells are activated and expanded for about 6 hours, about 12 hours, about 18 hours or about 24 hours prior to introduction of a nucleic acid (e.g., expression vector) encoding the cell surface receptor the into the T cells. In some embodiments, T cells are activated at the same time that a nucleic acid (e.g., an expression vector) encoding the cell surface receptor is introduced into the T cells.

In some embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan. Further illustrative examples of cell culture media include, but are not limited to RPMI 1640, Clicks, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

In some embodiments, the nucleic acid (e.g., an expression vector) encoding the cell surface receptor is introduced into the cell (e.g., a T cell) by microinjection, transfection, lipofection, heat-shock, electroporation, transduction, gene gun, microinjection, DEAE-dextran-mediated transfer, and the like. In some embodiments, the nucleic acid (e.g., expression vector) encoding the cell surface receptor is introduced into the cell (e.g., a T cell) by AAV transduction. The AAV vector may comprise ITRs from AAV2, and a serotype from any one of AAV1, AAV2, AAV3, AAV4, AAV5, AAV6, AAV7, AAV8, AAV9, or AAV 10. In some embodiments, the AAV vector comprises ITRs from AAV2 and a serotype from AAV6. In some embodiments, the nucleic acid (e.g., expression vector) encoding the cell surface receptor is introduced into the cell (e.g., a T cell) by lentiviral transduction. The lentiviral vector backbone may be derived from HIV-1, HIV-2, visna-maedi virus (VMV) virus, caprine arthritis-encephalitis virus (CAEV), equine infectious anemia virus (EIAV), feline immunodeficiency virus (FIV), bovine immune deficiency virus (BIV), or simian immunodeficiency virus (SIV). The lentiviral vector may be integration competent or an integrase deficient lentiviral vector (TDLV). In one embodiment, IDLV vectors including an HIV-based vector backbone (i.e., HIV cis-acting sequence elements) are employed.

Also provided are viruses that include any of the recombinant polypeptides, nucleic acids, and/or expression vectors of the present disclosure.

### Compositions

Also provided are compositions comprising any of the recombinant polypeptides, proteases, nucleic acids, expression vectors, and/or cells described herein.

In some embodiments, the compositions include any of the recombinant polypeptides, proteases, nucleic acids, expression vectors, and/or cells of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazi ne-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

Pharmaceutical compositions are also provided. The pharmaceutical compositions may include any of the cells of the present disclosure, and a pharmaceutically acceptable carrier. The pharmaceutical compositions generally include a therapeutically effective amount of the cells. By “therapeutically effective amount” is meant a number of cells sufficient to produce a desired result, e.g., an amount sufficient to effect beneficial or desired therapeutic (including preventative) results, such as a reduction in a symptom of a disease (e.g., cancer) or disorder associated, e.g., with the target cell or a population thereof (e.g., cancer cells), as compared to a control. An effective amount can be administered in one or more administrations.

The cells of the present disclosure can be incorporated into a variety of formulations for therapeutic administration. More particularly, the cells of the present disclosure can be formulated into pharmaceutical compositions by combination with appropriate, pharmaceutically acceptable excipients or diluents.

Formulations of the cells suitable for administration to a patient (e.g., suitable for human administration) are generally sterile and may further be free of detectable pyrogens or other contaminants contraindicated for administration to a patient according to a selected route of administration.

The cells may be formulated for parenteral (e.g., intravenous, intra-arterial, intraosseous, intramuscular, intracerebral, intracerebroventricular, intrathecal, subcutaneous, etc.) administration, or any other suitable route of administration.

Pharmaceutical compositions that include the cells of the present disclosure may be prepared by mixing the cells having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

An aqueous formulation of the recombinant polypeptides, proteases, nucleic acids, expression vectors, and/or cells may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

In some embodiments, the pharmaceutical composition includes cells of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

In certain aspects, provided is a pharmaceutical composition that includes a therapeutically effective amount of cells (e.g., T cells, such as CAR T cells) of the present disclosure. A “therapeutically effective amount” of such cells may vary according to factors such as the disease state, age, sex, and weight of the individual, and the ability of the cells to elicit a desired response in the individual. A therapeutically effective amount is also one in which any toxic or detrimental effects of the cells are outweighed by the therapeutically beneficial effects. The term “therapeutically effective amount” includes an amount that is effective to “treat” an individual, e.g., a patient. When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (individual). In some embodiments, a pharmaceutical composition of the present disclosure includes from 1×10⁶ to 5×10¹⁰ of the cells of the present disclosure.

### Methods of Use

In another aspect, provided herein are methods that employ the recombinant polypeptides, proteases, nucleic acids, expression vectors, and/or cells described herein.

In certain embodiments, provided are methods of regulating cellular localization of a protein of interest. Such methods include contacting a cell that expresses the recombinant polypeptide and a protease (where the protease cleavage site is a cleavage site for the protease), with an inhibitor of the protease when retention of the protein of interest in the cellular compartment determined by the protein localization tag is desired. According to some embodiments, the cellular compartment determined by the protein localization tag is selected from ER, Golgi, lysosome, plasma membrane, mitochondria, peroxisome, cytosol, and nucleus.

According to some embodiments, the protein of interest is engineered. For example, the protein of interest may be an engineered receptor (e.g., a CAR, an engineered TCR, or the like), and the method comprises regulating cellular localization of the engineered receptor between the cellular compartment determined by the protein localization tag and the cell surface. The cellular compartment determined by the protein localization tag may be, e.g., ER, Golgi, or lysosome. In certain embodiments, the cellular compartment determined by the protein localization tag is the ER. According to some embodiments, the cellular compartment determined by the protein localization tag is the Golgi.

According to some embodiments, the protein of interest is a transcription factor, e.g., an engineered or non-engineered transcription factor. When the protein of interest is a transcription factor, in certain embodiments, the methods comprise regulating cellular localization of the transcription factor between the cellular compartment determined by the protein localization tag and the nucleus. The cellular compartment determined by the protein localization tag may be, e.g., ER, Golgi, or lysosome. In certain embodiments, the cellular compartment determined by the protein localization tag is the ER. According to some embodiments, the cellular compartment determined by the protein localization tag is the Golgi. In certain embodiments, the cellular compartment determined by the protein localization tag is the plasma membrane. According to some embodiments, the cellular compartment determined by the protein localization tag is the cytosol.

In certain embodiments, the protein of interest is a secreted effector molecule, non-limiting examples of which include a stimulatory ligand, an inhibitory ligand, a cytokine, a chemokine, a growth factor, and a protease. According to some embodiments, when the protein of interest is a secreted effector molecule, the protein localization tag is an ER localization tag. For example, as a result of the ER localization tag, the secreted effector molecule may be insoluble and positioned in the ER lumen in the presence of the protease inhibitor, and upon ceasing the contacting (e.g., withdrawal of the protease inhibitor), the secreted effector molecule is cleaved from the ER localization tag, becomes soluble in the ER lumen, and is secreted into the extracellular space.

In certain embodiments, the protease is derived from HCV NS3, and the inhibitor of the protease is selected from imeprevir, danoprevir, asunaprevir, grazoprevir, simeprevir, ciluprevir, boceprevir, sovaprevir, paritaprevir, telaprevir, and any combination thereof. The methods may be carried out in vitro or ex vivo (e.g., in cultured cells), or in vivo, e.g., in an individual in a therapeutic context, e.g., an individual receiving a regulatable cell-based therapy of the present disclosure. In some embodiments, the methods of regulating cellular localization of a protein of interest further include ceasing the contacting when retention of the protein of interest in the cellular compartment determined by the protein localization tag is no longer desired.

In some embodiments, provided are methods of administering a regulatable cell-based therapy (e.g., a CAR T cell-based therapy) to an individual in need thereof. In some embodiments, the individual in need thereof has cancer, and the protein of interest (e.g., CAR) binds to a molecule on the surface of the cancer cells. The methods of administering a regulatable cell-based therapy to the individual include administering to the individual a pharmaceutical composition that includes cells that regulatably express any of the cell surface receptor proteins of interest of the present disclosure (CARs, TCRs, etc.) on the cell surface upon cleavage of the protein localization tag from the cell surface receptor protein of interest in the absence of an inhibitor of the protease. The pharmaceutical composition typically includes a therapeutically effective amount of such cells as described above. The cells may be any cells capable of effecting the desired therapy. In some embodiments, the cells are immune cells. Non-limiting examples of immune cells which may be administered include T cells, B cells, natural killer (NK) cells, macrophages, monocytes, neutrophils, dendritic cells, mast cells, basophils, and eosinophils. In certain embodiments, the cells are T cells. According to some embodiments, the cells are T cells and the protein of interest is a CAR, such that the cells are CAR T cells. In certain embodiments, the cells are stem cells, e.g., embryonic stem cells or adult stem cells. In some embodiments, the pharmaceutical composition is an autologous composition produced by a method including removing cells from the individual and introducing into the removed cells or progeny thereof the desired nucleic acid or expression vector.

The methods of administering a regulatable cell-based therapy to an individual may further include contacting the administered cells or progeny thereof with an inhibitor of the protease when retention of the protein of interest at the cellular compartment determined by the protein localization tag is desired, where the contacting includes administering the inhibitor of the protease to the individual. Retention of the protein of interest at the cellular compartment determined by the protein localization tag may be desired for a variety of reasons. For example, in the case of CAR T cells where the protein of interest is a CAR, retention of the CAR at the cellular compartment determined by the protein localization tag (as opposed to being expressed and functional/active on the surface of the cell) may be desirable to prevent or delay the onset of cell exhaustion (e.g., T cell exhaustion) resulting from CAR activity. As such, the inhibitor of the protease may be administered to prevent or delay the onset of cell exhaustion resulting from CAR activity. As another example, retention of the CAR at the cellular compartment determined by the protein localization tag may be desired in order to reduce adverse side effects caused by the cells or progeny thereof, e.g., side effects relating to activity of the CAR expressed on the surface of the cells or progeny thereof.

Contacting the administered cells or progeny thereof with the protease inhibitor may include administering to the individual an amount of the inhibitor effective to inhibit the protease. As just one example, when the protease is derived from HCV NS3 as described elsewhere herein, the contacting may include administering to the individual by a suitable route of administration simeprevir, danoprevir, asunaprevir, ciluprevir, boceprevir, sovaprevir, paritaprevir, telaprevir, grazoprevir, or any combination thereof, in an amount effective to inhibit the protease expressed by the administered cells or progeny thereof. According to the methods of administering a regulatable cell-based therapy to an individual, the inhibitor of the protease may be administered to the individual prior to, concurrently with (that is, co-administered), and/or subsequent to administration of the pharmaceutical composition to the individual.

The methods of administering a regulatable cell-based therapy to an individual may further include ceasing administration of the protease inhibitor when retention of the protein of interest at the cellular compartment determined by the protein localization tag is no longer desired.

In some embodiments, administration of the protease inhibitor is regulated in such a manner to yield a receptor (e.g., CAR) activation profile that: (1) promotes persistence of the cells that include the receptor (e.g., CAR-T cells); (2) promotes the formation of memory T cells (T_{HEM}), T memory stem cells (T_{SCM}), central memory T cells (T_{CM}), and/or effector memory T cells (T_{EM}); (3) promotes long-term functionality and proliferative potential of T cells; and/or reduces activation induced cell death (AICD) of T cells. The beneficial effects of providing CAR-T cells with periods of rest are described, e.g., in Viaud et al. (2018) PNAS 115(46):E10898-E10906.

According to the methods of administering a regulatable cell-based therapy (e.g., CAR T cell-based therapy), the methods may include administering the pharmaceutical composition to the individual under conditions in which the protease inhibitor is withheld to allow cell surface expression (and activity/signaling) of the cell surface receptor on the cells or progeny thereof in the individual, and subsequently administering the protease inhibitor when cell surface expression (and activity/signaling) of the cell surface receptor on the cells or progeny thereof is no longer desired. Cell surface expression (and activity/signaling) may no longer be desired for one or more reasons. For example, expression of a CAR on the surface of T cells may no longer be desired in order to delay or prevent cell exhaustion resulting from CAR signaling. Accordingly, the methods may include administering the protease inhibitor to delay or prevent cell exhaustion resulting from CAR activity. In some embodiments, T cell exhaustion resulting from cell surface expression of the CAR may be due to antigen-independent tonic signaling and/or prolonged antigen-dependent signaling through antigen engagement. Alternatively, or additionally, cell surface expression of the CAR may no longer be desired because of adverse side effects caused by the cells or progeny thereof, such that the methods may include administering the protease inhibitor to reduce adverse side effects caused by the cells or progeny thereof. Adverse side effects may include, but are not limited to, off tumor effects (e.g., on-target, off-tumor activity of the CAR), toxicity resulting from, e.g., unrestricted antigen-driven proliferation of the cells, and the like. Such toxicity may include cytokine release syndrome and/or neurotoxicity. Accordingly, in some embodiments, the methods may further include administering the protease inhibitor to reduce adverse side effects caused by the cells or progeny thereof.

In some embodiments, cell surface expression of the receptor (e.g., a CAR) is regulated in order to optimize the activation profile of the cells that include the receptor, e.g., CAR T cells. Optimizing the activation profile finds use, e.g., for retaining high functionality and persistence. For example, with respect to CAR T cells, “always on” CAR-T cells may tend to have a higher fraction of short-lived effector T cell subsets, whereas regulated CAR-T cells may be tuned so that they have a higher fraction of long-lived memory T cell subsets. During their manufacture, regulated CAR T cells may also be able to undergo more rounds of expansion than unregulated CAR-T cells.

In some embodiments, the amount of cell surface expression of the receptor is tuned by selecting a protease cleavage site having a particular “strength” (where a “stronger” cleavage site is cleaved by the protease more efficiently than a “weaker” cleavage site is cleaved by the protease), the amount of the protease inhibitor administered to the individual, or a combination thereof. By way of example, when the protease is derived from HCV NS3, non-limiting examples of protease cleavage sites having varying strengths are those comprising the amino acid sequences set forth in SEQ ID Nos:31-35.

When the protease is derived from HCV NS3, the methods of administering a regulatable cell-based therapy to an individual may include administering an effective amount of a protease inhibitor selected from imeprevir, danoprevir, asunaprevir, grazoprevir, simeprevir, ciluprevir, boceprevir, sovaprevir, paritaprevir, telaprevir, and any combination thereof, when retention of the protein of interest in the cellular compartment determined by the protein localization tag is desired.

The methods of administering a regulatable cell-based therapy to an individual may further include producing the pharmaceutical composition. Producing the pharmaceutical composition may include introducing an expression vector of the present disclosure into cells or progeny thereof obtained from the individual (e.g., to produce an autologous composition) or into cells obtained from a donor (e.g., to produce an allogeneic composition).

### Kits

Also provided by the present disclosure are kits. In certain embodiments, provided are kits that include any of the nucleic acids and/or expression vectors of the present disclosure, and instructions for introducing the nucleic acid or expression vector into a cell. According to some embodiments, when the expression vector encodes a recombinant polypeptide that does not comprise the protease (trans configuration), the expression vector further encodes the protease. In certain embodiments, the expression vector is configured to express the recombinant polypeptide and the protease from the same promoter. For example, the expression vector may be a bicistronic expression vector for expression of separate recombinant polypeptides and protease molecules under the same promoter in the cell.

The kits find use in a variety of in vitro, ex vivo, and in vivo applications. The instructions of such kits may further include instructions for regulating cellular localization of the protein of interest. For example, the instructions of such kits may further include instructions for contacting the cell or progeny thereof with an inhibitor of the protease when retention of the protein of interest in the cellular compartment determined by the protein localization tag is desired. The instructions of such kits may further include instructions for ceasing the contacting when retention of the protein of interest in the cellular compartment determined by the protein localization tag is no longer desired.

The kits of the present disclosure may further include any other reagents useful for regulatable signaling of the cell surface receptor, such as transfection/transduction reagents useful for introducing the nucleic acid or expression vector into cells of interest, e.g., immune cells (e.g., T cells) or other cells of interest.

In some embodiments, the kits further include an inhibitor of the protease. For example, when a protease derived from HCV NS3 as described elsewhere herein is employed, the kit may include a suitable inhibitor of the protease, including but not limited to, imeprevir, danoprevir, asunaprevir, grazoprevir, simeprevir, ciluprevir, boceprevir, sovaprevir, paritaprevir, telaprevir, or any combination thereof.

Components of the kits may be present in separate containers, or multiple components may be present in a single container. A suitable container includes a single tube (e.g., vial), one or more wells of a plate (e.g., a 96-well plate, a 384-well plate, etc.), or the like.

The instructions of the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### EXPERIMENTAL

### Example 1—Requlatable Cellular Localization of Cell Surface Receptors

In this example, recombinant polypeptides in which the protein of interest is a CAR and the recombinant polypeptides include a protease cleavage site, protease and protein localization tag (sometimes referred to herein as a “STASH tag”) were tested for the ability to regulate cellular localization of the CAR. The recombinant polypeptides were as schematically illustrated in FIG. 1, panel A, but did not include the green fluorescent protein (GFP) domain.

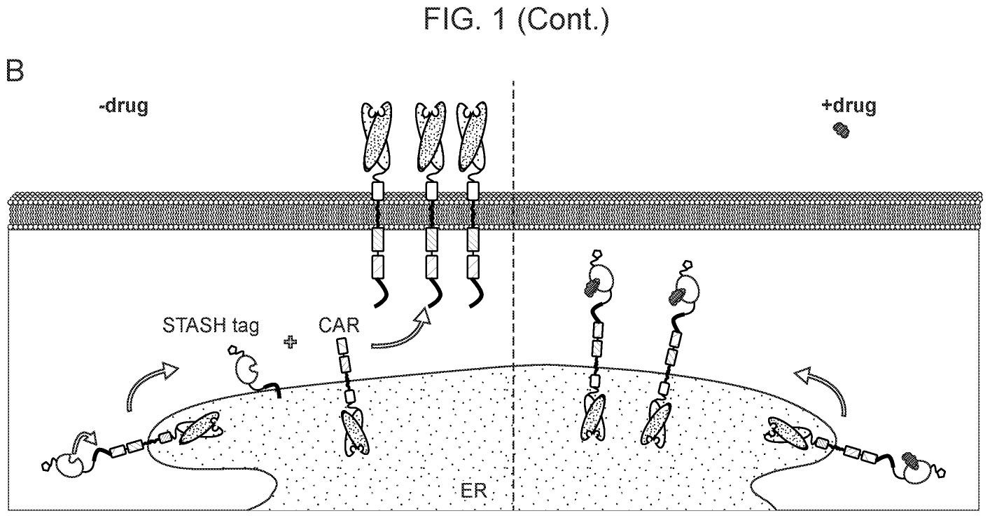

Shown in FIG. 2 is a graph showing the quantification of CAR surface molecules on primary human T cells by flow cytometry for a panel of B7H3 CAR-STASH designs bearing various retention signals and tested in the presence (+) or absence (−) of HCV NS3 protease inhibitor (3 μM grazoprevir). Surface CAR molecules were stained using fluorescently-labeled B7H3-Fc. The recombinant polypeptides designated “95”, “96”, “97” and “98” comprise the amino acid sequences set forth in SEQ ID Nos: 36, 37, 38 and 39, respectively. As is apparent in FIG. 2, cell surface expression of the CAR was substantially greater in the absence of the protease inhibitor as compared to the presence of the protease inhibitor. In other words, the reduced cleavage of the localization tag in the presence of the protease inhibitor enabled retention of the CAR at the cellular compartment determined by the protein localization tag, whereas the protein localization tag was cleaved from the CAR in the absence of the inhibitor, thereby enabling cell surface expression of the CAR.

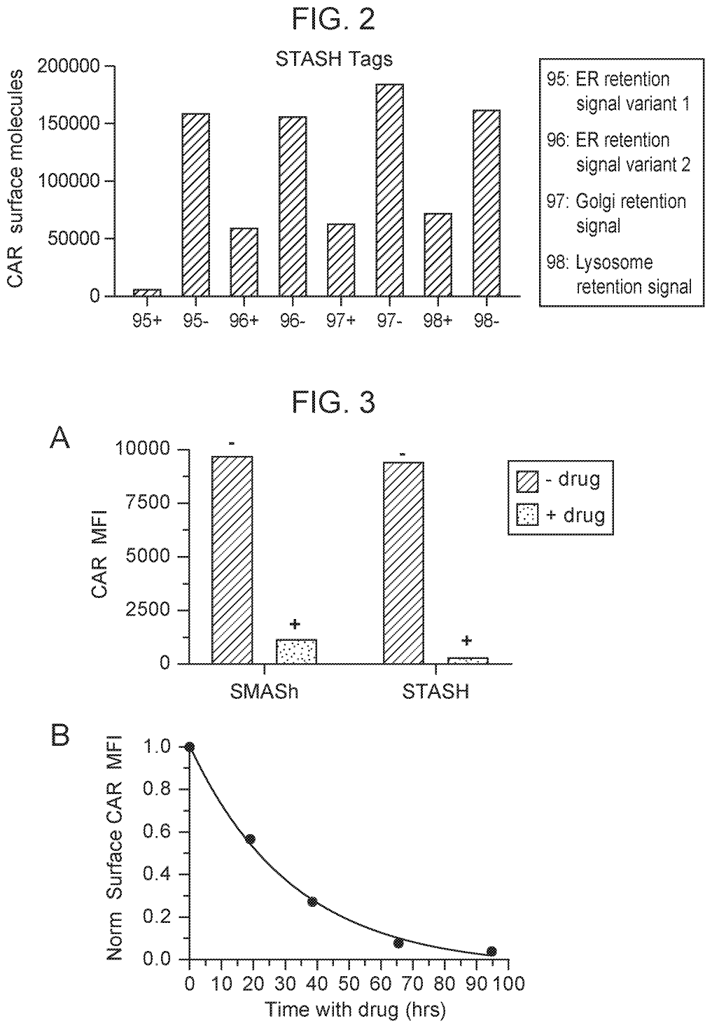

Shown in FIG. 3, panel A, is a graph showing the quantification of CAR surface molecules on primary human T cells by flow cytometry for a B7H3 CAR-SMASh and B7H3 CAR-STASH (ER) tested in the presence (+) or absence (−) of HCV NS3 protease inhibitor (3 μM grazoprevir). CAR Surface molecules were stained using fluorescently-labeled B7H3-Fc. By “SMASh” is meant the CAR is tagged with a tag comprising the protease, a degron (a sequence that directs degradation of the CAR unless cleaved from the CAR, and a cleavage site for the protease disposed between the CAR and the degron. See International Application No. PCT/US2019/040572. Shown in FIG. 3, panel B, is a graph showing the reduction of B7H3 CAR surface expression after incubation with drug (3 μM grazoprevir) for various lengths of time.

The data demonstrate that the CAR constructs with ER retention tag exhibit high surface expression in the absence of drug, low surface expression in the presence of drug, and rapidly reduced surface expression after addition of drug.

### Example 2—CAR Localization by Fluorescence Microscopy and Flow Cytometry

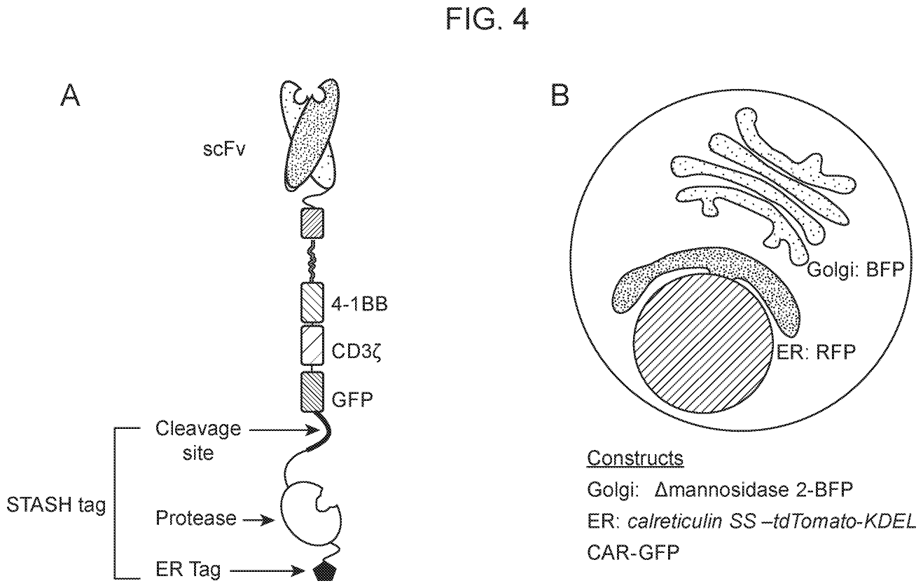

In this example, recombinant polypeptides in which the protein of interest is a CAR and the recombinant polypeptides include a fluorescent reporter (GFP) domain and a STASH tag—as schematically illustrated in FIG. 4, panel A—were visualized by fluorescence microscopy. FIG. 4, panel B, is a schematic of a 293T cell that is labeled with fluorescently tagged proteins that localize to various cellular compartments.

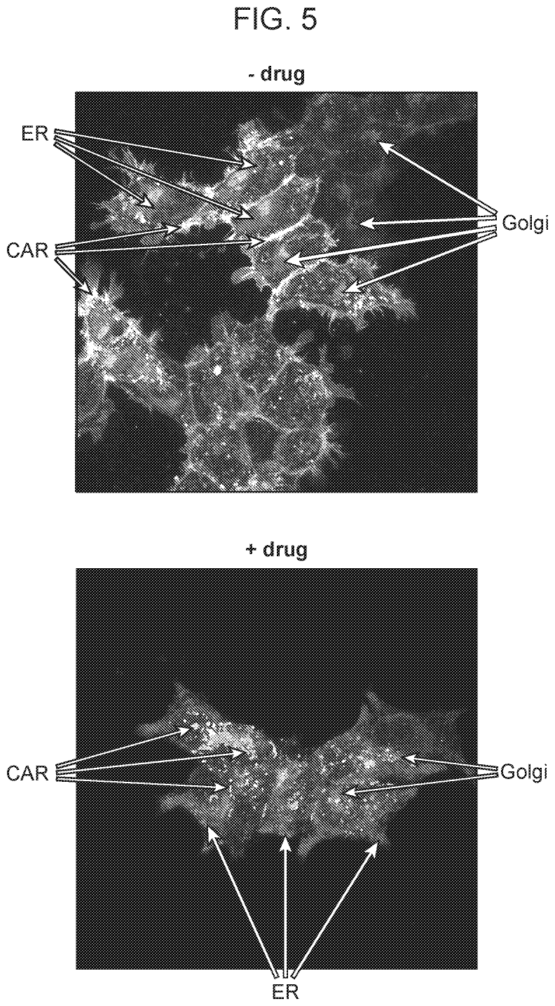

FIG. 5 shows microscopy images of 293T cells transduced with B7H3-STASH (ER) and incubated in the absence (−drug) or presence (+drug) of 3 μM grazoprevir. As can be seen in the images, B7H3-STASH ER molecules which are labeled with GFP (green) are primarily expressed on the cell surface in the absence of drug, whereas the CAR molecules are primarily stored in intracellular compartments in the presence of drug.

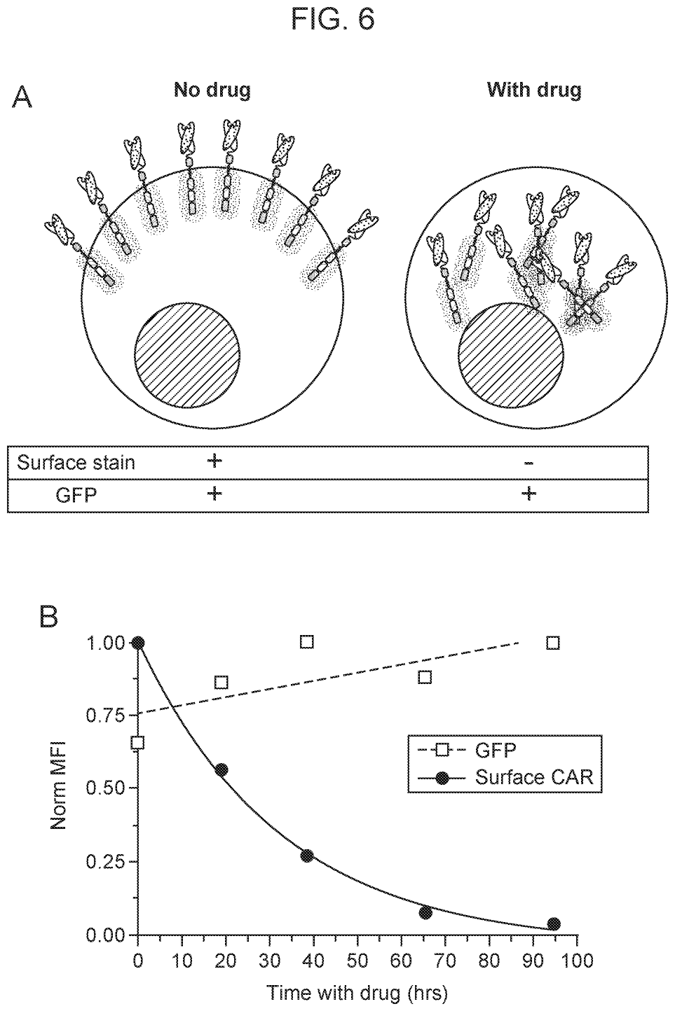

Next, flow cytometry was employed to assess intracellular retention (rather than degradation) of CAR molecules in the absence of the protease inhibitor. FIG. 6, panel A, is a schematic showing CAR-GFP-STASH molecules localized to the surface or intracellularly and their corresponding staining, where “+” represents positive staining and “−” represents negative staining. FIG. 6, panel B, is a plot of the quantification of flow cytometry data for B7H3-CAR-STASH T cells incubated with drug (3 μM grazoprevir) for various amounts of time. As can be seen in the plot, CAR surface staining diminishes after incubation with drug, whereas the GFP signal, which can be detected irrespective of the localization of GFP, remains relatively constant. These data indicate that the CAR molecules are retained intracellularly, not merely degraded, after incubation with drug.

### Example 3— STASH CAR-T Cells Secrete IFNγ During Coculture with Tumor Cells

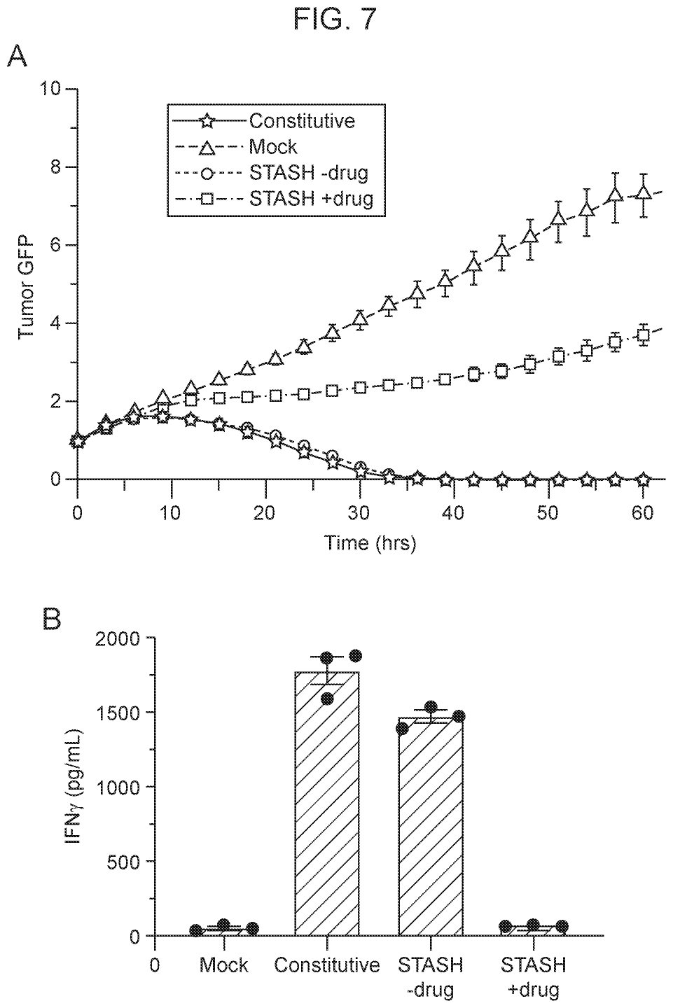

Shown in FIG. 7, panel A, is a graph showing the GFP fluorescence of GFP-expressing D425 medulloblastoma cells which also express the B7H3 antigen. The tumor cells were co-cultured with B7H3-CAR-STASH T cells in the presence (+drug) or absence (−drug) of 3 μM grazoprevir. The cytotoxic capacity of B7H3-CAR-STASH T cells can be controlled by the addition of drug, as determined by tumor GFP fluorescence. Constitutive B7H3 CAR T cells lacking the STASH tag and Mock untransduced T cells serve as positive and negative controls, respectively.

Shown in FIG. 7, panel B, is a graph showing quantification of interferon gamma (IFNγ) levels in co-culture supernatant taken from co-cultures described in FIG. 7, panel A. As can be seen in panel B, the level of IFNγ secreted in co-cultures can be controlled by the addition of drug.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A recombinant polypeptide comprising:
a protein of interest;
a protein localization tag; and
a protease cleavage site disposed between the protein of interest and the protein localization tag.

2. The recombinant polypeptide of claim 1, comprising from N-terminus to C-terminus:
the protein of interest;
the protease cleavage site; and
the protein localization tag.

3. The recombinant polypeptide of claim 1, comprising from N-terminus to C-terminus:
the protein localization tag
the protease cleavage site; and
the protein of interest.

4. The recombinant polypeptide of any one of claims 1 to 3, wherein the protein localization tag is selected from the group consisting of: an endoplasmic reticulum (ER) localization tag, a Golgi apparatus (Golgi) localization tag, a lysosome localization tag, a plasma membrane localization tag, a mitochondria localization tag, a peroxisome localization tag, a cytosolic localization tag, and a nuclear localization tag.

5. The recombinant polypeptide of any one of claims 1 to 4, wherein the protein localization tag is an ER localization tag.

6. The recombinant polypeptide of claim 5, wherein the ER localization tag comprises 85% or greater, 90% or greater, or 100% amino acid sequence identity to the amino acid sequence LYKYKSRRSFIDEKKMP (SEQ ID NO:1).

7. The recombinant polypeptide of claim 5, wherein the ER localization tag comprises the amino acid sequence KKMP (SEQ ID NO:2).

8. The recombinant polypeptide of any one of claims 1 to 4, wherein the protein localization tag is a Golgi localization tag.

9. The recombinant polypeptide of claim 8, wherein the Golgi localization tag comprises the amino acid sequence YQRL (SEQ ID NO:24).

10. The recombinant polypeptide of any one of claims 1 to 4, wherein the protein localization tag is a lysosome localization tag.

11. The recombinant polypeptide of claim 10, wherein the lysosome localization tag comprises the amino acid sequence KFERQ (SEQ ID NO:25).

12. The recombinant polypeptide of any one of claims 1 to 11, wherein the protein of interest is not engineered.

13. The recombinant polypeptide of any one of claims 1 to 11, wherein the protein of interest is engineered.

14. The recombinant polypeptide of claim 13, wherein the engineered protein of interest is an engineered cell surface receptor.

15. The recombinant polypeptide of claim 14, wherein the engineered cell surface receptor is a chimeric antigen receptor (CAR).

16. The recombinant polypeptide of claim 15, wherein the CAR comprises a first and a second intracellular signaling domain or fragments thereof independently selected from the group consisting of: a CD3 intracellular signaling domain, a CD28 intracellular signaling domain, a 4-1 BB intracellular signaling domain, an OX-40 intracellular signaling domain, an inducible co-stimulator (ICOS) intracellular signaling domain, an CD27 intracellular signaling domain, and a MyD88/CD40 intracellular signaling domain.

17. The recombinant polypeptide of claim 16, wherein the first intracellular signaling domain or fragment thereof is a CD3 intracellular signaling domain and the second intracellular signaling domain or fragment thereof is a 4-1 BB intracellular signaling domain.

18. The recombinant polypeptide of claim 16, wherein the first intracellular signaling domain or fragment thereof is a CD3 intracellular signaling domain and the second intracellular signaling domain or fragment thereof is a CD28 intracellular signaling domain.

19. The recombinant polypeptide of any one of claims 15 to 18, wherein the extracellular binding domain of the CAR comprises a single chain antibody.

20. The recombinant polypeptide of claim 19, wherein the single chain antibody is a single chain variable fragment (scFv).

21. The recombinant polypeptide of claim 19 or claim 20, wherein the extracellular binding domain of the CAR specifically binds an antigen expressed on the surface of a cancer cell.

22. The recombinant polypeptide of claim 21, wherein the cancer cell-surface antigen is selected from the group consisting of: B7-H3 (CD276), CD19, GD2, CD22, and HER2.

23. The recombinant polypeptide of claim 13, wherein the engineered protein of interest is a T cell receptor (TCR).

24. The recombinant polypeptide of claim 23, wherein the TCR recognizes an antigen complexed with a major histocompatibility complex (MHC) molecule displayed on the surface of a cancer cell.

25. The recombinant polypeptide of any one of claims 1 to 13, wherein the protein of interest is a cell surface molecule.

26. The recombinant polypeptide of claim 25, wherein the cell surface molecule is a cell surface receptor.

27. The recombinant polypeptide of claim 25 or claim 26, wherein the cell surface molecule is selected from the group consisting of: a cytokine receptor, a chemokine receptor, an adhesion molecule, an integrin, an inhibitory receptor, an inhibitory cell surface ligand, a stimulatory receptor, a stimulatory cell surface ligand, an immunoreceptor tyrosine-based activation motif (ITAM)—containing receptor, and an immunoreceptor tyrosine-based inhibition motif (ITIM)—containing receptor.

28. The recombinant polypeptide of any one of claims 1 to 13, wherein the protein of interest is a transcription factor.

29. The recombinant polypeptide of any one of claims 1 to 13, wherein the protein of interest is a secreted effector molecule.

30. The recombinant polypeptide of claim 29, wherein the secreted effector molecule is selected from the group consisting of: a stimulatory ligand, an inhibitory ligand, a cytokine, a chemokine, a growth factor, and a protease.

31. The recombinant polypeptide of claim 29 or claim 30, wherein the protein localization tag is an ER localization tag.

32. The recombinant polypeptide of any one of claims 1 to 31, wherein the protease cleavage site is a viral protease cleavage site.

33. The recombinant polypeptide of claim 32, wherein the viral protease cleavage site is for a viral protease derived from hepatitis C virus (HCV) nonstructural protein 3 (NS3).

34. The recombinant polypeptide of claim 33, wherein the viral protease cleavage site is for a viral protease that further comprises a cofactor polypeptide derived from HCV nonstructural protein 4A (NS4A).

35. The recombinant polypeptide of any one of claims 32 to 34, wherein the viral protease cleavage site is selected from the group consisting of: an NS4A/4B junction cleavage site, an NS3/NS4A junction cleavage site, an NS4A/NS4B junction cleavage site, an NS4B/NS5A junction cleavage site, an NS5A/NS5B junction cleavage site, and variants thereof cleavable by the viral protease.

36. The recombinant polypeptide of any one of claims 1 to 35, further comprising a reporter domain.

37. The recombinant polypeptide of claim 36, wherein the reporter domain comprises a fluorescent protein.

38. The recombinant polypeptide of claim 37, wherein the fluorescent protein is green fluorescent protein.

39. The recombinant polypeptide of claim 36, wherein the reporter domain comprises a bioluminescent protein.

40. The recombinant polypeptide of claim 39, wherein the bioluminescent protein is a luciferase.

41. The recombinant polypeptide of claim 40, wherein the luciferase is a nanoluciferase.

42. The recombinant polypeptide of any one of claims 36 to 41, wherein the reporter domain is disposed between the protein of interest and the protease cleavage site.

43. The recombinant polypeptide of any one of claims 1 to 42, wherein the recombinant polypeptide further comprises a protease, and wherein the protease cleavage site is a cleavage site for the protease.

44. The recombinant polypeptide of claim 43, wherein the protease cleavage site is disposed between the protein of interest and the protease.

45. The recombinant polypeptide of claim 43 or claim 44, comprising from N-terminus to C-terminus:
the protein of interest;
the protease cleavage site;
the protease; and
the protein localization tag.

46. The recombinant polypeptide of claim 43 or claim 44, comprising from N-terminus to C-terminus:
the protein localization tag;
the protease
the protease cleavage site; and
the protein of interest.

47. A nucleic acid encoding the recombinant polypeptide of any one of claims 1 to 46.

48. An expression vector comprising the nucleic acid of claim 47.

49. A cell comprising the nucleic acid of claim 47 or the expression vector of claim 48.

50. The cell of claim 49, wherein the cell is a mammalian cell.

51. The cell of claim 50, wherein the cell is a human cell.

52. The cell of any one of claims 49 to 51, wherein the cell is an immune cell.

53. The cell of claim 52, wherein the immune cell is selected from the group consisting of: a T cell, a B cell, a natural killer (NK) cell, a macrophage, a monocyte, a neutrophil, a dendritic cell, a mast cell, a basophil, and an eosinophil.

54. The cell of claim 53, wherein the immune cell is a T cell.

55. The cell of claim 54, wherein the T cell is selected from the group consisting of: a naive T cell (T_{N}), a cytotoxic T cell (T_{CTL}), a memory T cell (T_{HEM}), a T memory stem cell (T_{SCM}), a central memory T cell (T_{CM}), an effector memory T cell (T_{EM}), a tissue resident memory T cell (T_{RM}), an effector T cell (T_{EFF}), a regulatory T cell (T_{REGs}), a helper T cell, a CD4+ T cell, a CD8+ T cell, a virus-specific T cell, an alpha beta T cell (Tαβ), and a gamma delta T cell (T_{γδ).}

56. The cell of claim 54 or claim 55, wherein the protein of interest is a CAR.

57. The cell of claim 56, wherein the protein of interest is the CAR of the recombinant polypeptide of any one of claims 15 to 22.

58. The cell of claim 54, wherein the protein of interest is a TCR.

59. The cell of any one of claims 49 to 58, wherein the nucleic acid encodes the recombinant polypeptide of any one of claims 43 to 46.

60. The cell of any one of claims 49 to 58, wherein the recombinant polypeptide does not comprise a protease.

61. The cell of claim 60, further comprising a nucleic acid encoding a protease, wherein the protease cleavage site is a cleavage site for the protease.

62. The cell of claim 61, wherein the nucleic acid encoding the protease is present on the expression vector of claim 48.

63. The cell of claim 61, wherein the nucleic acid encoding the protease is present on an expression vector other than the expression vector of claim 48.

64. The cell of any one of claims 61 to 63, wherein the protease is a soluble cytosolic protease.

65. The cell of any one of claims 61 to 63, wherein the protease is expressed on the cytosolic side of the cellular compartment determined by the protein localization tag.

66. The cell of any one of claims 61 to 63, wherein the protease is expressed on the lumen side of the cellular compartment determined by the protein localization tag.

67. A method of making the cell of any one of claims 49 to 66, comprising introducing the nucleic acid of claim 47 or the expression vector of claim 48 into the cell.

68. A method of making the recombinant polypeptide of any one of claims 1 to 46, comprising culturing a cell comprising the expression vector of claim 48 under conditions in which the cell expresses the recombinant polypeptide.

69. A method of regulating cellular localization of a protein of interest, comprising:
contacting a cell that expresses:
the recombinant polypeptide of any one of claims 1 to 42; and
a protease, wherein the protease cleavage site is a cleavage site for the protease,
with an inhibitor of the protease when retention of the protein of interest at the cellular compartment determined by the protein localization tag is desired.

70. The method according to claim 69, wherein the cellular compartment determined by the protein localization tag is selected from the group consisting of: ER, Golgi, lysosome, plasma membrane, mitochondria, peroxisome, cytosol, and nucleus.

71. The method according to claim 69 or claim 70, wherein the protein of interest is engineered.

72. The method according to claim 71, wherein the engineered protein of interest is an engineered receptor.

73. The method according to claim 72, wherein the method comprises regulating cellular localization of the engineered receptor between the cellular compartment determined by the protein localization tag and the cell surface.

74. The method according to claim 72 or claim 73, wherein the cellular compartment determined by the protein localization tag is selected from the group consisting of: ER, Golgi, and lysosome.

75. The method according to any one of claims 72 to 74, wherein the engineered receptor is a CAR.

76. The method according to any one of claims 72 to 74, wherein the engineered receptor is a TCR.

77. The method according to any one of claims 69 to 71, wherein the protein of interest is a transcription factor.

78. The method according to claim 77, wherein the method comprises regulating cellular localization of the transcription factor between the cellular compartment determined by the protein localization tag and the nucleus.

79. The method according to claim 78, wherein the cellular compartment determined by the protein localization tag is the plasma membrane.

80. The method according to claim 78, wherein the cellular compartment determined by the protein localization tag is the cytosol.

81. The method according to claim 78, wherein the cellular compartment determined by the protein localization tag is selected from the group consisting of: ER, Golgi, and lysosome.

82. The method according to any one of claims 69 to 71, wherein the protein of interest is a secreted effector molecule.

83. The method according to claim 82, wherein the secreted effector molecule is selected from the group consisting of: a stimulatory ligand, an inhibitory ligand, a cytokine, a chemokine, a growth factor, and a protease.

84. The method according to claim 82 or claim 83, wherein the protein localization tag is an ER localization tag.

85. The method according to claim 84, wherein the secreted effector molecule is insoluble and positioned in the ER lumen in the presence of the protease inhibitor.

86. The method according to claim 85, wherein upon ceasing the contacting, the secreted effector molecule becomes soluble in the ER lumen and is secreted into the extracellular space.

87. The method according to any one of claims 69 to 81, wherein the protease is derived from HCV NS3, and wherein the inhibitor of the protease is selected from the group consisting of: asunaprevir (ASV), danoprevir (DPV), simeprevir (SPV), grazoprevir (GPV), and any combination thereof.

88. The method according to any one of claims 69 to 87, further comprising ceasing the contacting when retention of the protein of interest at the cellular compartment determined by the protein localization tag is no longer desired.

89. The method according to any one of claims 69 to 88, wherein the method is performed in vitro.

90. The method according to any one of claims 69 to 88, wherein the method is performed ex vivo.

91. The method according to any one of claims 69 to 88, wherein the method is performed in vivo.

92. The method according to any one of claims 69 to 91, wherein the recombinant polypeptide comprises the protease.

93. The method according to any one of claims 69 to 91, wherein the recombinant polypeptide does not comprise the protease.

94. A pharmaceutical composition comprising:
the cell of any one of claims 49 to 66; and
a pharmaceutically-acceptable carrier.

95. A method of making the pharmaceutical composition of claim 94, comprising introducing the expression vector of claim 48 into cells obtained from an individual.

96. A method of administering a regulatable cell-based therapy to an individual in need thereof, comprising administering to the individual the pharmaceutical composition of claim 94, wherein the cells express a protease, and wherein the protease cleavage site is a cleavage site for the protease.

97. The method according to claim 96, wherein the recombinant polypeptide comprises the protease.

98. The method according to claim 96, wherein the recombinant polypeptide does not comprise the protease.

99. The method according to any one of claims 96 to 98, further comprising administering to the individual an inhibitor of the protease when retention of the protein of interest at the cellular compartment determined by the protein localization tag is desired.

100. The method according to claim 99, wherein the inhibitor of the protease is administered concurrently with the pharmaceutical composition.

101. The method according to claim 99 or claim 100, wherein the inhibitor of the protease is administered subsequently to administration of the pharmaceutical composition.

102. The method according to any one of claims 99 to 101, further comprising ceasing administration of the protease inhibitor when retention of the protein of interest at the cellular compartment determined by the protein localization tag is no longer desired.

103. The method according to any one of claims 99 to 102, wherein the protease is derived from HCV NS3, and wherein the protease inhibitor is selected from the group consisting of: asunaprevir (ASV), danoprevir (DPV), simeprevir (SPV), grazoprevir (GPV), and any combination thereof.

104. The method according to any one of claims 96 to 103, wherein the pharmaceutical composition comprises immune cells comprising the expression vector of claim 48.

105. The method according to claim 104, wherein the immune cells are selected from the group consisting of: T cells, B cells, natural killer (NK) cells, macrophages, monocytes, neutrophils, dendritic cells, mast cells, basophils, and eosinophils.

106. The method according to claim 105, wherein the immune cells are T cells.

107. The method according to claim 106, wherein the protein of interest is a CAR.

108. The method according to claim 106, wherein the protein of interest is a TCR.

109. A kit, comprising:
the expression vector of claim 48; and
instructions for introducing the expression vector into a cell.

110. The kit of claim 109, wherein the instructions further comprise instructions for regulating cellular localization of the protein of interest.

111. The kit of claim 110, wherein the instructions comprise instructions for contacting the cell or progeny thereof with an inhibitor of the protease when retention of the protein of interest at the cellular compartment determined by the protein localization tag is desired.

112. The kit of claim 111, wherein the instructions comprise instructions for ceasing the contacting when retention of the protein of interest at the cellular compartment determined by the protein localization tag is no longer desired.

113. The kit of any one of claims 109 to 112, further comprising an inhibitor of the protease.


SEQUENCE LISTING (40 sequences)

SEQ ID NO: 1 - protein, 17 aa, Artificial sequence
  synthetic polypeptide
LYKYKSRRSFIDEKKMP

SEQ ID NO: 2 - protein, 4 aa, Artificial sequence
  synthetic polypeptide
KKMP

SEQ ID NO: 3 - protein, 6 aa, Artificial sequence
  synthetic polypeptide
AEKDEL

SEQ ID NO: 4 - protein, 14 aa, Artificial sequence
  synthetic polypeptide
EQKLISEEDLKDEL

SEQ ID NO: 5 - protein, 14 aa, Artificial sequence
  synthetic polypeptide
GGGGSGGGGSKDEL

SEQ ID NO: 6 - protein, 24 aa, Artificial sequence
  synthetic polypeptide
GGGGSGGGGSGGGGSGGGGSKDEL

SEQ ID NO: 7 - protein, 26 aa, Artificial sequence
  synthetic polypeptide
GGGGSGGGGSGGGGSGGGGSAEKDEL

SEQ ID NO: 8 - protein, 15 aa, Artificial sequence
  synthetic polypeptide
KYKSRRSFIEEKKMP

SEQ ID NO: 9 - protein, 16 aa, Artificial sequence
  synthetic polypeptide
LKYKSRRSFIEEKKMP

SEQ ID NO: 10 - protein, 17 aa, Artificial sequence
  synthetic polypeptide
LYKYKSRRSFIEEKKMP

SEQ ID NO: 11 - protein, 18 aa, Artificial sequence
  synthetic polypeptide
LYCKYKSRRSFIEEKKMP

SEQ ID NO: 12 - protein, 19 aa, Artificial sequence
  synthetic polypeptide
LYCNKYKSRRSFIEEKKMP

SEQ ID NO: 13 - protein, 17 aa, Artificial sequence
  synthetic polypeptide
LYKYKSRRSFIDEKKMP

SEQ ID NO: 14 - protein, 19 aa, Artificial sequence
  synthetic polypeptide
LYCNKYKSRRSFIDEKKMP

SEQ ID NO: 15 - protein, 27 aa, Artificial sequence
  synthetic polypeptide
LYEQKLISEEDLKYKSRRSFIEEKKMP

SEQ ID NO: 16 - protein, 27 aa, Artificial sequence
  synthetic polypeptide
LYCYPYDVPDYAKYKSRRSFIEEKKMP

SEQ ID NO: 17 - protein, 12 aa, Artificial sequence
  synthetic polypeptide
LYKKLETFKKTN

SEQ ID NO: 18 - protein, 22 aa, Artificial sequence
  synthetic polypeptide
LYEQKLISEEDLKKLETFKKTN

SEQ ID NO: 19 - protein, 6 aa, Artificial sequence
  synthetic polypeptide
LYYQRL

SEQ ID NO: 20 - protein, 16 aa, Artificial sequence
  synthetic polypeptide
LYEQKLISEEDLYQRL

SEQ ID NO: 21 - protein, 30 aa, Artificial sequence
  synthetic polypeptide
LYKRKIIAFALEGKRSKVTRRPKASDYQRL

SEQ ID NO: 22 - protein, 8 aa, Artificial sequence
  synthetic polypeptide
LYRNIKCD

SEQ ID NO: 23 - protein, 18 aa, Artificial sequence
  synthetic polypeptide
LYEQKLISEEDLRNIKCD

SEQ ID NO: 24 - protein, 4 aa, Artificial sequence
  synthetic polypeptide
YQRL

SEQ ID NO: 25 - protein, 5 aa, Artificial sequence
  synthetic polypeptide
KFERQ

SEQ ID NO: 26 - protein, 15 aa, Artificial sequence
  synthetic polypeptide
GGGGSGGGGSGGGGS

SEQ ID NO: 27 - protein, 186 aa, hepatitis C virus
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 28 - protein, 186 aa, hepatitis C virus
APITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTD

SEQ ID NO: 29 - protein, 186 aa, hepatitis C virus; identical to SEQ ID NO: 27

SEQ ID NO: 30 - protein, 186 aa, hepatitis C virus
APITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 31 - protein, 9 aa, hepatitis C virus
DEMEECSQH

SEQ ID NO: 32 - protein, 9 aa, hepatitis C virus
DEMEECSQH

SEQ ID NO: 33 - protein, 9 aa, hepatitis C virus
EDVVPCSMG

SEQ ID NO: 34 - protein, 10 aa, hepatitis C virus
EDVVPCSMGS

SEQ ID NO: 35 - protein, 11 aa, hepatitis C virus
ECTTPCSGSWL

SEQ ID NO: 36 - protein, 732 aa, Artificial sequence
  synthetic polypeptide
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGGGSGGSDEMEECSQHGGSGGSTGCVVIVGRIVLSGSGTSAPITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHGGSGGSLYKYKSRRSFIDEKKMP

SEQ ID NO: 37 - protein, 719 aa, Artificial sequence
  synthetic polypeptide
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGGGSGGSDEMEECSQHGGSGGSTGCVVIVGRIVLSGSGTSAPITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHGGSGGSKKMP

SEQ ID NO: 38 - protein, 719 aa, Artificial sequence
  synthetic polypeptide
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGGGSGGSDEMEECSQHGGSGGSTGCVVIVGRIVLSGSGTSAPITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHGGSGGSYQRL

SEQ ID NO: 39 - protein, 720 aa, Artificial sequence
  synthetic polypeptide
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGGGSGGSDEMEECSQHGGSGGSTGCVVIVGRIVLSGSGTSAPITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHGGSGGSKFERQ

SEQ ID NO: 40 - protein, 362 aa, Artificial sequence
  synthetic polypeptide
ACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSTGCVVIVGRIVLSGSGTSAPITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTH